(19) United States
(12) Patent Application Publication
(10) Pub. No.: US 2024/0158774 A1
(43) Pub. Date: May 16, 2024

(54) Compositions and Methods for Treating Celiac Sprue Disease

(71) Applicant: UNIVERSITY OF WASHINGTON THROUGH ITS CENTER FOR COMMERCIALIZATION, Seattle, WA (US)

(72) Inventors: Justin Bloomfield SIEGEL, Seattle, WA (US); David BAKER, Seattle, WA (US); Ingrid Swanson PULTZ, Seattle, WA (US)

(21) Appl. No.: 18/357,617

(22) Filed: Jul. 24, 2023

Related U.S. Application Data
(63) Continuation of application No. 17/230,230, filed on Apr. 14, 2021, now abandoned.
Application No. 17/230,230 is a continuation of application No. 16/693,057, filed on Nov. 22, 2019, now Pat. No. 11,008,558.
Application No. 16/693,057 is a continuation of application No. 16/360,190, filed on Mar. 21, 2019, now Pat. No. 10,487,318.
Application No. 16/360,190 is a continuation of application No. 14/911,630, filed on Feb. 11, 2016, now Pat. No. 10,266,815.
(60) Provisional application No. 61/865,787, filed on Aug. 14, 2013.

(51) Int. Cl. C12N 9/52; C12N 9/64

(52) U.S. Cl. CPC C12N 9/52; C12N 9/64; C12N 9/6489; A61K 38/00

(57) ABSTRACT

The invention provides compositions and methods for treating celiac sprue.

### RELATED APPLICATIONS

This application claims priority to U.S. Provisional Patent Application Ser. No. 61/865,787 filed Aug. 14. 2013, incorporated by reference herein in its entirety.

### FEDERAL FUNDING STATEMENT

This invention was made with government support under HR0011-08-1-0085 awarded by the Defense Advanced Research Projects Agency. The government has certain rights in the invention.

### BACKGROUND

Celiac sprue is a highly prevalent disease in which dietary proteins found in wheat, barley, and rye products known as ‘glutens’ evoke an immune response in the small intestine of genetically predisposed individuals. The resulting inflammation can lead to the degradation of the villi of the small intestine, impeding the absorption of nutrients. Symptoms can appear in early childhood or later in life, and range widely in severity, from diarrhea, fatigue and weight loss to abdominal distension, anemia, and neurological symptoms. There are currently no effective therapies for this lifelong disease except the total elimination of glutens from the diet. Although celiac sprue remains largely underdiagnosed, its' prevalence in the US and Europe is estimated at 0.5-1.0% of the population. The identification of suitable naturally-occurring enzymes as oral therapeutics for celiac disease is difficult due to the stringent physical and chemical requirements to specifically and efficiently degrade gluten-derived peptides in the harsh and highly acidic environment of the human digestive tract.

### SUMMARY OF THE INVENTION

In one aspect, the invention provides methods for treating celiac sprue, comprising administering to a subject with celiac sprue an amount effective to treat the celiac sprue of one or more polypeptides comprising or consisting of the amino acid sequence of a polypeptide selected from the group consisting of SEQ ID NOS: 75, 74, 76-89, 95, 97-98, 102-111, or processed versions thereof. In one embodiment, the one or more polypeptides comprise one or more polypeptides comprising or consisting of the amino acid sequence of a polypeptide selected from the group consisting of SEQ ID NOs: 75, 74, 77, 78, 82, 88, 98, 105, 111, or processed versions thereof. In a further embodiment, the one or more polypeptides comprise a polypeptide with the amino acid sequence of SEQ ID NO: 89.

In another aspect, the invention provides isolated polypeptides selected from the group consisting of the following polypeptides, or processed versions thereof:
(a) SEQ ID NO: 95, wherein one, two, three, four, five, six, seven, eight, nine, ten, or all eleven of the following are true: (i) AA residue 116 is V or D; (ii) AA residue 255 is S, K, or G; (iii) AA residue 284 is D; (iv) AA residue 285 is T; (v) AA residue 286 is A, T, or N; (vi) AA residue 312 is S; (vii) AA residue 347 is N; (viii) AA residue 350 is T or A; (ix) AA residue 351 is N or G; (x) AA residue 354 is D; and (xi) AA residue 361 is S or H;
(b) SEQ ID NO: 75, wherein one, two, three, four, five, six, seven, eight, nine, ten, or all eleven of the following are true: (i) AA residue 106 is D; (ii) AA residue 246 is S, K, or G; (iii) AA residue 275 is D; (iv) AA residue 276 is S; (v) AA residue 277 is A, T, or N; (vi) AA residue 303 is S; (vii) AA residue 338 is S; (viii) AA residue 341 is T or A; (ix) AA residue 342 is N or G; (x) AA residue 345 is Q or D; and (xi) AA residue 352 is S or H;
(c) SEQ ID NO: 76, wherein one, two, three, four, five, six, seven, eight, nine, ten, or all eleven of the following are true: (i) AA residue 120 is D; (ii) AA residue 259 is S, K, or G; (iii) AA residue 288 is D; (iv) AA residue 289 is T; (v) AA residue 290 is A, T, or N; (vi) AA residue 316 is S; (vii) AA residue 351 is S or N; (viii) AA residue 354 is A; (ix) AA residue 355 is N or G; (x) AA residue 358 is D; and (xi) AA residue 365 is S or H;
(d) SEQ ID NO: 78;
(e) SEQ ID NO: 79, wherein one, two, three, four, five, six, seven, eight, nine, ten, or all eleven of the following are true: (i) AA residue 107 is V or D; (ii) AA residue 245 is S, K, or G; (iii) AA residue 274 is D; (iv) AA residue 275 is T; (v) AA residue 276 is A, T, or N; (vi) AA residue 302 is S; (vii) AA residue 337 is S or N; (viii) AA residue 340 is T or A; (ix) AA residue 341 is N or G; (x) AA residue 344 is Q or D; and (xi) AA residue 351 is S or H;
(f) SEQ ID NO: 80, wherein one, two, three, four, five, six, seven, eight, nine, ten, or all eleven of the following are true: (i) AA residue 76 is V or D; (ii) AA residue 206 is S, K, or G; (iii) AA residue 235 is D; (iv) AA residue 236 is S; (v) AA residue 237 is A, T, or N; (vi) AA residue 262 is S; (vii) AA residue 297 is S or N; (viii) AA residue 300 is T or A; (ix) AA residue 301 is N or G; (x) AA residue 302 is Q or D; and (xi) AA residue 309 is S or H;
(g) SEQ ID NO: 81, wherein one, two, three, four, five, six, seven, eight, nine, ten, or all eleven of the following are true: (i) AA residue 105 is D; (ii) AA residue 244 is S or K; (iii) AA residue 272 is D; (iv) AA residue 273 is S; (v) AA residue 274 is A, T, or N; (vi) AA residue 299 is S; (vii) AA residue 334 is N; (viii) AA residue 337 is I or A; (ix) AA residue 338 is N or G; (x) AA residue 341 is Q or D; and (xi) AA residue 348 is S or H;
(h) SEQ ID NO: 82, wherein one, two, three, four, five, six, seven, eight, nine, ten, or all eleven of the following are true: (i) AA residue 106 is V or D; (ii) AA residue 244 is S, K, or G; (iii) AA residue 273 is D; (iv) AA residue 274 is T; (v) AA residue 275 is A, T, or N; (vi) AA residue 301 is S; (vii) AA residue 336 is N; (viii) AA residue 339 is T or A; (ix) AA residue 340 is N or G; (x) AA residue 343 is D; and (viii) AA residue 350 is S or H;
(I) SEQ ID NO: 83, wherein one, two, three, four, five, six, seven, eight, nine, ten, or all eleven of the following are true: (i) AA residue 107 is V or D; (ii) AA residue 245 is S, K or G; (iii) AA residue 274 is D; (iv) AA residue 275 is T; (v) AA residue 276 is A, T, or N; (vi) AA residue 302 is S; (vii) AA residue 337 is N; (viii) AA residue 340 is T or A; (ix) AA residue 341 is N or G; (x) AA residue 344 is Q or D; and (xi) AA residue 351 is S or H;
(j) SEQ ID NO: 84, wherein one, two, three, four, five, six, seven, eight, nine, ten, or all eleven of the following are true: (i) AA residue 104 is V or D; (ii) AA residue 241 is S, K, or G; (iii) AA residue 270 is D; (iv) AA residue 271 is S; (v) AA residue 272 is D, A, T, or N; (vi) AA residue 398 is S; (vii) AA residue 33 is S; (viii) AA residue 336 is A; (ix) AA residue 337 is N or G; (x) AA residue 340 is D; and (xi) AA residue 347 is S or H;
(k) SEQ ID NO: 85, wherein one, two, three, four, five, six, seven, eight, nine, ten, or all eleven of the following are true: (i) AA residue 104 is D; (ii) AA residue 245 is S, K, or G; (iii) AA residue 274 is D; (iv) AA residue 275 is S; (v) AA residue 276 is A, T, or N; (vi) AA residue 302 is S; (vii) AA residue 337 is S or N; (viii) AA residue 340 is T or A; (ix) AA residue 341 is N or G; (x) AA residue 344 is Q or D; and (xi) AA residue 351 is S or H;
(l) SEQ ID NO: 86, wherein one, two, three, four, five, six, seven, eight, nine, ten, or all eleven of the following are true: (i) AA residue 118 is V or D; (ii) AA residue 250 is K, or G; (iii) AA residue 279 is D; (iv) AA residue 280 is S; (v) AA residue 281 is A, T, or N; (vi) AA residue 307 is S; (vii) AA residue 342 is S or N; (viii) AA residue 345 is A; (ix) AA residue 346 is N or G; (x) AA residue 349 is Q or D; and (xi) AA residue 356 is S or H;
(m) SEQ ID NO: 87, wherein one, two, three, four, five, six, seven, eight, nine, ten, or all eleven of the following are true: (i) AA residue 121 is V or D; (ii) AA residue 253 is S, K, or G; (iii) AA residue 282 is D; (iv) AA residue 283 is S; (v) AA residue 284 is A, T, or N; (vi) AA residue 310 is S; (vii) AA residue 345 is S; (viii) AA residue 348 is T or A; (ix) AA residue 349 is N or G; (x) AA residue 352 is Q or D; and (xi) AA residue 357 is S or H;
(n) SEQ ID NO: 88, wherein one, two, three, four, five, six, seven, eight, nine, or all ten of the following are true: (i) AA residue 111 is S, K, or G; (ii) AA residue 139 is D; (iii) AA residue 140 is T or S; (iv) AA residue 141 is D, A, T, or N; (v) AA residue 164 is S; (vi) AA residue 199 is S or N; (vii) AA residue 202 is T or A; (viii) AA residue 203 is N or G; (ix) AA residue 204 is Q or D; and (x) AA residue 211 is S or H; and
(o) SEQ ID NO:89.

In other aspects, the invention provides nucleic acids encoding the polypeptides of the invention, nucleic acid expression vectors comprising the isolated nucleic acids of the invention, and recombinant host cells comprising the nucleic acid expression vectors of the invention. of

[symbol]

In a still further aspect, the invention provides compositions, comprising
(a) one or more polypeptides comprising the amino acid sequence of a polypeptide selected from the group consisting of SEQ ID NOs: 74-89, 95, 97-99, and 102-111, or processed versions thereof; and
(b) one or more further polypeptides comprising an amino acid sequence selected from the group consisting of:
   (A) an amino acid sequence at least 75% identical to the amino acid sequence of SEQ Ill NO:35, wherein
   (i) the polypeptide degrades a PQPQLP (SEQ ID NO:34) peptide at pH 4; and
   (ii) residue 278 is Ser, residue 78 is Glu, and residue 82 is Asp
   (B) an an amino acid sequence at least 75% identical to the amino acid sequence of SEQ IO NO:1, wherein
   (i) the polypeptide degrades a PQPQLP (SEQ IIS NO:34) peptide at pH 4; and
   (ii) residue 467 is Ser, residue 267 is Glu, and residue 271 is Asp.

In a further aspect, the invention provides pharmaceutical compositions, comprising the isolated polypeptides, nucleic acids, expression vectors, host cells, or compositions of the invention, together with a pharmaceutically acceptable carrier.

### DESCRIPTION OF THE FIGURES

FIG. 1 is a graph showing the activity of various polypeptides to break down a fluorescent analogue of gliadin that was conjugated to a fluorophore and a quencher. X-axis: specific homologue number, KWT=Kumamolisin-As. Y axis: arbitrary enzyme units.

FIG. 2 is a graph showing polypeptide activity in breaking down a fluorescent analogue of gliadin at various pH levels. SC PEP: prolyl endopeptidase from Sphingomonas capsulata. WT: Kumamolisin-As. Max: KumaMax™. Blue bars represent optimal protease activity at the indicated pH level.

FIG. 3 is a graph showing the activity of various polypeptides to break down two peptides that are degradation products of gluten. On the X-axis is time in minutes, and the Y-axis is fraction of peptide remaining, Max=KumaMax™; K3=Kumamolisin-As active site mutant; H4M=Homologue 4 (SEQ ID NO: 75) KumaMax™-mutant; 33=33mer peptide (SEQ ID NO: 72); 26=26mer peptide (SEQ ID NO: 73).

FIG. 4 is a graph showing the activity of various additional polypeptides to break down the 33mer peptide degradation product of gluten (SEQ ID NO: 72).

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2^{nd} Ed. (R. I. Freshney. 1987. Liss, Inc. New York, NY), Gene Transfer and Expression Protocols, pp. 109-128, ed. E. J. Murray, The Humana Press Inc., Clifton, N.J.), and the Ambion 1998 Catalog (Ambion, Austin, TX).

As used herein, the singular forms “a”, “an” and “the” include plural referents unless the context clearly dictates otherwise. “And” as used herein is interchangeably used with “or” unless expressly stated otherwise.

As used herein, amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the invention can be used in combination, unless the context clearly dictates otherwise.

In a first aspect, the present invention provides methods for treating celiac sprue, comprising administering to a subject with celiac sprue an amount effective to treat the celiac sprue of one or more polypeptides comprising or consisting of the amino acid sequence of a polypeptide selected from the group consisting of the following, or processed versions thereof:

Homologue 1
(SEQ ID NO: 74)
MAPSDVEIVDPVAPEERITVTVLLRRRSSIPDQLIEGPDTLSRAELADRHGADPADVEAVRVAMSGAGLTVVGTDLPSRRV
TVAGTAEALMRTFGAELQIVRDASGFQHRARSGELRIPAALDGIVIAVLGLDNRPQAEARFRASQPEAARSFRPDALGRVY
RFPANTDGTGQTIAIVELGGGFRQSELDTYFGGLGIPAPQVLAVGVDGGQNLPSGDAGSADGEVLLDIEVAGALAPGARQV
VYFAPNTDRGFVDAVTTAVHADPTPAAVSISWGAPEDKWTAQARRAFDAALADAAALGVTVTAAAGDRGSADGEGGGGLHT
DFPASSPHLLACGGTKLAVADGGTVASETVWNGGERGGATGGGVSVAFGLPAYQRNAGVDKRRKTGKPGRGVPDVAAVADP
ATGYEVLVDGEQLVFGGTSAVAPLWAALVARLTQALGRPLGLLNTALYDGAQPGRTQPGFRDVTEGDNDISGKHGPYPARA
GWDACTGLGVPDGEALLAALRKPGKE;
Homologue 2
(SEQ ID NO: 95)
MQRGTKEGLNMARHLQADREPRIVPESKCLGQCDPAERIHVTIMLRRQEEGQLDALVHQLATGDARAKPVSRDAFAQRFSA
NPDDIRKTEDFAHRHQLTVDRVDPVESVVVLSGT(I/V/D)AQFEAAFSVKLERFEHRSIGQYRGRSGPIVLPDDIGDAVT
AVLGLDSRPQARPHFRFRPPFKPARGAAAVTFTPIQLASLYDFPAGDGAGQCIAIIELGGGYRAADIQQYFRGLGITTPPK
LVDVNVGTGRNAPTGEP(N/S/K/G)GPDGEVALDIEIAGAIAPAAKIAVYFAP(N/D)(S/T)(D/A/T/N)AGFIQAVN
AAVTDKTNQPSVISISW(G/S)GPEAIWQAQSAQAFNRVLQAAAAQGITVCAASGD(S/N)GS(G/T/A)(D/N/G)GL
(Q/D)DGADHV(D/S/H)FPASSPYVLGCGGTQLDALPGQGIRSEVTWNDEASGGGAGGGGVSALFDLPAWQQGLKVARAD
GTTTPLAKRGVPDVAGDASPQTGYEVSVAGTPAVMGGTSAVAPLWAALIARINAANGASAGWINPVLYKHPGALRDITKGS
NGTYAAASGWDACTGLGSPNGAQLATILARKPSS;
Homologue 4
(SEQ ID NO: 75)
MANHPLNGSERECLKDAQPIGKADPNERLEVTMLVRRRSHDAFEKHISALAAQGASAKHIDHDEFTKHFGADSADLAAVHA
FAQKHGLSVVESHEARRAVVLSGT(V/D)AQFDAAFGVSLQQYEHDGGTYRGRTGPIHLPDELNGVVDAVMGLDNRPQARP
SFRTRAQGNVRWTARAAGASTFTPVQLASLYDFPQGDGQNQCIGIIELGGGYRPADLKTYFASLNMKAPSVTAVSVDHGRN
HPTGDP(N/S/K/G)GPDGEVMLDIEVAGAVAPGAKIVVYFAP(N/D)(T/S)(D/A/T/N)AGFIDAIGTAIHDTKNKPS
VISISW(G/S)GPESAWTQQAMNAFDQAFQSAAALGVTICAASGD(N/S)GS(G/T/A)(D/N/G)GV(G/Q/D)DGADHV
(D/S/H)FPASSPYALGCGGTSLQASGNGIASETVWNDGANGGATGGGVSSFFALPAWQEGLRVTRAGGAHSPLAMRGVPD
VAGNADPVTGYEVRVDGHDMVIGGTSAVAPLWAGLIARINAIKGAPVGYINPHLYKDPLALVDITKGNNDDFHATAGWDAC
TGLGRPDGKKVKDAVS;
Homologue 5
(SEQ ID NO: 76)
MNHDHSPTGGELSNWVRVPGSERAAVQGSRKVGPADPNEQMSVTVVVRRPAADTAVTSMIEKVGAQPLSERRHLTREEFAS
THGANPADLSKVEKFAHEHNLQVKEVNAAAGTMVLSGT(V/D)TSFSKAFGVELSTYEHPDFTYRGRIGHVHIPDYLADTI
QSVLGLDNRPQASPRFRVLKEEGGVTTAHAGRTSYTPLEVAALYNFPSIHCKDQCIGILELGGGYRPADLQTYFNGLGIPQ
PNITDVSVGGAANRPTGDP(N/S/K/G)GPDGEVVLDIEVAAAVTPGAKIAVYFAD(N/D)(S/T)(D/A/T/N)DGFLNA
ITTAIHDTRNKPSVISISW(G/S)KAEIGWTPQAINAMNQAFRDAAALGVTICCASGD(D/S/N)GS(T/A)(D//N/G)R
V(Q/D)DGRYHV(D/S/H)FPASSPYVLACGGTRLESSGSTITQEVVWNEGALGGGATGGGVSDVFDRPNWQANANVPTSA
NPERRIGRGVPDWAGNADPATGYQILVDGTRAVIGGTSAVAPLFAGLIAIINQKLGHSVGFINPILYNLSAQHNVFHDITS
GNNDMSGQNGPYEAQPGWDACTGLGSPDGTKLMNAISEAHRLVSVG;
Homologue 6
(SEQ ID NO: 77)
MAPEERRTLPGSAMPRPAGAQVLGQIPDDERVEVTVVLQPRAPLPEPGPTPMSRAELADLRSPPEGALEAIARYVAGQGLE
VIAADAPRRRIVLAGSAARIAALFGISFVRLQLEGRRYRTYEGEISLPAELAPLVVAVLGLDTRPFARSHRRPAVAPNAPT
TAPTVARAYDFPTAYDGRGTTIGFIELGGGFQESDLVRYCEGLGLSTPQVSVVGVDGARNAPTGDPNGPDAEVMLDLEVAT
GVANGADLVLYMAANTDAAFYSAIATALRDATHAPVAISISWGAPEESYPATTIAAFESVLEEAVHVGVTVLVAAGDQGST
DGVDDGRAHVDYPAASPYVLACGGTRLDLDGTTIVAETVWNDLPNGGATGGGISALFPVPSWQAGIAMPPSANPGAGPGRG
VPDVAGNADPDTGYRIVVDGVATVVGGTSAVAPLWAGLVARCHQAGARGGFWNPLLYAARGSSAFHEITVGSNGAYDAGPI
WNACCGLGSPNGTAILQTLRA;
Homologue 6 mutant:
(SEQ ID NO: 78)
MAPEERRTLPGSAMPRPAGAQVLGQIPDDERVEVTVVLQPRAPLPEPGPTPMSRAELADLRSPPEGALEAIARYVAGQGLE
VIAADAPRRRIVLAGSAARIAALFGISFVRLQLEGRRYRTYEGEISLPAELAPLVVAVLGLDTRPFARSHRRPAVAPNAPT
TAPTVARAYDFPTAYDGRGTTIGFIELGGGFQESDLVRYCEGLGLSTPQVSVVGVDGARNAPTGDPNGPDAEVMLDLEVAT
GVANGADLVLYMAANTDAAFYSAIATALRDATHAPVAISISWSAPEESYPATTIAAFESVLEEAVHVGVTVLVAAGDQGST
GGVDDGRAHVHYPAASPYVLACGGTRLDLDGTTIVAETVWNDLPNGGATGGGISALFPVPSWQAGIAMPPSANPGAGPGRG
VPDVAGNADPDTGYRIVVDGVATVVGGTSAVAPLWAGLVARCHQAGARGGFWNPLLYAARGSSAFHEITVGSNGAYDAGPI
WNACCGLGSPNGTAILQTLRA;
Homologue 9
(SEQ ID NO: 79)
MTKQPVSGSSDKIHPDDAKCIGDCDPSEQIEVIVMLRRKDEAGFRQMMSRIDAGEAPGQAVSREEFDRRFTASDEDIDKVK
AFAKQYGLSVERAETETRSVVLKGT(I/V/D)EQFQKAFDVKLERFQHHNIGEYRGRTGPVNVPDEMHDAVTAVLGLDSKP
QARPHFRFRPPFKPLRGAAPASFSPVDLAKLYDFPDGDGAGQCIAIIELGGGYRDSDLSAYFSKLGVKAPTVVPVGVDGGK
NAPTGNP(N/S/K/G)GPDGEVTLDIEIAGAIAPGARIAVYFAP(N/D)(S/T)(D/A/T/N)AGFVDAVNRALHDAANKP
SVISISW(G/S)GPESNWSPQSMSAFNDVLQSAAALGVTVCAASGD(G/S/N)GS(A/T)(D/N/G)GV(G/Q/D)DGADH
V(D/S/H)FPASSPYVLGCGGTSLAASGAGIAKEVVWNDGDQGGAGGGGVSGTFALPVWQKGLSVTRNGKHIALAKRGVPD
VAGDASPQTGYEVLIDGEDTVVGGTSAVAPLWAALIARINAIDASPAGFVNPKLYKAKTAFRDITEGNNGSFSAAAGWDAC
TGMGSPDGGKIAAALKPAKPSQSAGQQ;
Homologue 10
(SEQ ID NO: 80)
MGRLQGSYRPSLGTPVGPVPDDQPIDVTVVLRPTAADDFRADPDDVAAVRAFAGRAGLDVAEVDEPARTVRLRGP(AN/D)
AAARTAFDTPLALYDSGGRAIRGREGDLGLPDELDDRVVAVLGLDERPAARPRFQPAASARQGLTALQVARAYDFPAATGE
GQTIAIIELGGGFGQADLDTYFGGLDLPTPAVSAVGVQGAANVPGGDP(/S/K/G)DGADGEVLLDIEVAGAVAPGAAQVV
YFAP(N/D)(T/S)(D/A/T/N)AGFLAAINAAAAATPRPAAISISWG(G/S)PESSWTAQAMRAYDQAFAAARAAGITVL
AAAGD(A/S/N)GA(D/T/A)(D/S/N/G)(A/Q/D)TDRLVA(D/S/H)FPAGSPNVIACGGTKLTLDAAGARASEVVWN
EAADSATGGGYSATFTRPAWQPAAVGRYRGLPDISGNADPQTGYRVVVDGQPTVVGGTSAVAPLLAGLVARLAQLTGAPVA
DLAAVAYANPAAFTDITAGDNQGYPARSGWDPASGLGSPVGTKLLTAVGGPTPPPTTPPPTTPPPTTPPPTIPPPTTPPTQ
TVDAADRALWSAVATWAGGTHTGANARAAKAVRAWAQAKSLA;
Homologue 12
(SEQ ID NO: 81)
MTQPRYTPLPGSEREAPLLAARSNATAARASRAQTASATVVLRRRSELPEALVLDQQFISSDELAARYGADPVDIEKVRSV
LERFKVSVVEVDAASRRVKVEGA(V/D)ADIERAFNIALHSASGTDPHSGRGFEYRYRTGVLSVPAELGGIVTAVLGLDNR
RQAETRLRVVPAAALGSSYTPVQLGEIYNFPQDATGAGQRIAIIELGGGYTPAGLRRYFASLGVVPPKVAAVSVDGAQNAP
GPDP(G/S/K/G)ADGEVQLDVEVAGALAPGAHVLVYFAP(N/D)(T/S)(D/A/T/N)QGFLDAVSQAAHATPPPTAISI
SW(G/S)ASEDSWTASARDALNQALRDAAALGVTVTAAAGD(S/N)GS(S/T/A)(D/N/G)GV(P/Q/D)DRRAHV(D/S
/H)FPASSPYVLATGGTSLRADPATGVVQSETVWNDSQGSTGGGVSDVFPRPAWQAHVDVPHAGRGVPDVSAVADPATGYQ
VLVDNQPAVIGGTSAVAPLWAALVARLAESLGRPLGLLQPLVYPRTPGSTAYPGFRDITIGNNGAYKAGKGWDAATGLGVP
DGTELLAHLRGLNGSE;
Homologue 13
(SEQ ID NO: 82)
MARHLHAGSEPKVITESKCIGACDPAERIHVTVMLRREGEQALDALVDKLASGDPAAKPVSREDFAKRFGARADDIQHTEA
FAKRHQLTVERVDPVQSVVELAGT(I/V/D)AQFENAFGVKLEKYEHHAIGSFRARTGAIALPDELHDAVTAVLGLDTRPQ
AHPHFRFRPPFQPARSGAGTSYTPLQLASIYNFPEGDGAGQCIALVELGGGYRAADIRQYFEQLGVKPPKLVDVSVNGGRN
APTDDP(N/S/K/G)GPDGEVALDIEVAGAIAPGATIAVYFAG(N/D)(S/T)(D/A/T/N)AGFIQSVNQAIHDSTNRPS
VVSISW(G/S)GPEASWTQQSITAFNNVLKTAASLGVTVCAASGD(S//N)GS(S/T/A)(D/N/G)GL(Q/D)DGSNHV
(D/S/H)FPASSPYVLACGGTTLDAQAGQGIRREVVWNDEAASGGAGGGGVSAVFPAPSYQKGLSAKATGGGSTPLSQRGV
PDVAGDASPTTGYIISIAGTTAVLGGTSAVAPLWAALIARINANGKSPVGWANPKLYAQPGAFHDITQGNNGAFAASEGWD
ACTGLGSPDGAKVAAALQGASGGSQQGRATGA;
Homologue 14
(SEQ ID NO: 83)
MTKHPLPGSERVLAPGSKVVAQCDPSETIEVVVVLRRKNEQQFAQMMKTIEAGAAGARPLTREELEQRFGALPEDIAKLKA
FAAQHGLSVVREDASARTVVLSGR(I/V/D)EQFQQAFDVQLQHYEHQSMGRFRGRTGAISVPDELHGVVTAVLGLDDRPQ
ARPHFRIRPPFQPARAQSASSFTPLQLASLYRFPQGDGSGQCIGIVELGGGYRTADLDSYFSSLGVGSPKVVAVGVDQSGN
QPTGDP(N/S/K/G)GPDGEVTLDIEIAGALAPAATIAVYFTT(N/D)(S/T)(D/A/T/N)AGFIDAVSQAVHDRTNQPS
VISISW(G/S)APESMWTAQSMKALNDVLQSAAAIGVTVCAASGD(S/N)GS(S/T/A)(D/N/G)GV(G/Q/D)DGRDHV
(D/S/H)FPASSPYVLACGGTSLQGSGRTVAHEVVWNDGSNGGATGGGVSGAFPVPAWQEGLSTSAAQGGQRALTGRGVPD
VAGDASPLTGYDVIVDGNNTVIGGTSAVAPLWAALIARINGAKGAPVGFVNPKLYKASACNDITQGNNGSYAATTGWDACT
GLGSPDGVKVAAAL;
Homologue 15
(SEQ ID NO: 84)
MSPIASRRSALPLSERPAPENARALAAVEPDRTMTVSVLVRRKKPLVLADLEGKKLTHREFERRYGASEKDFATIAKFAAG
HGLAVDHHASSLARRTVVLRGT(A/V/D)RQMQQAFGVTLHDYEDSETQQRYHSFTGAITVPAAHARIIESVLGLDARPIA
KPHFRVRKRSAAATGAVSFNPPQVASLYSFPTGVDGSGETIGILELGGGYETSDIQQYFSGLGIQPPTVVAVSVDGAVNAP
GNP(N/S/K/G)GADGEVALDIQVAGSIAPGAKLAVYFAP(N/D)(T/S)(E/D/A/TN)QGFVDAITTAVHDTANKPSVL
SISW(G/S)GPESSWPQAAAQSLNNACESAAALGVTITVASGD(N/S)GS(T/A)(D/N/G)GV(Q/D)DGQNHV(D/S/
H)FPASSPYVLACGGTYLAAVNNGVPQESVWDDLASGGGATGGGVSALFPLPAWQTGANVPGGSMRGVPDVAGDASPESGY
NVLVDGQPQVVGGTSAVAPLWAALIALVNQQKGEAAGFVNAALYQNPSAFHDITQGSNGAYAAAPGWDPCTGLGSPMGTAI
AKILA;
Homologue 16
(SEQ ID NO: 85)
MSAFDQLVPLPGSEKTVPDAAPSQTLDPNEVLTVTIRIRRKRTLASLVSTTAPVTEVVSRSEYASRFGADPAIVKQVEAFA
SAYDLSLVEQSLARRSVLLRGT(V/D)AQMEQAFGVSLANYQLADGTVFRGRTGVVNVPSELVEHIEGVFGLDNRPQARAH
FQVYKPEKGTKVAPRAGGISYTPPQLARLYNFPTGVTGKGQCIAIIELGGGFRTADIKTYFGGLGLKPPTVVAVSVDGGHN
APSTA(D/S/K/G)SADGEVMLDIDVAGGVAPGAKIVVYFAP(N/D)(T/S)(D/A/TN)QGFLDAITTAMHDTKNKPSVI
SISW(G/S)AAESNWTPQALTSFNQAFQAAAALGITVCAAAGD(T/S/N)GS(D/T/A)(D/N/G)SV(G/Q/D)DGKAHV
(D/S/H)FPASSPFVLACGGTKLTATDNVIASEVVWHESKTSATGGGVSDVFDLPDYQQKSHVPPSVNDKTRIGRGVPDVA
AVADPVTGYAVRVDGSNLVFGGTSAVAPLMAGLIALINQQRGKAVGFIHPLIYANPSAFRDITQGNNTTTTGNKGYAATTG
WDACTGLGVADGKKLASVLTATPVA;
Homologue 17
(SEQ ID NO: 86)
MAATPRFASQPRVTLPGSQKHPLTTDTEVPPPAPVKAAATKLSATPFTVTVIVKRKNPLNLKQVLKPAGRLTHAAFAKAHG
PSPDGVKLVKAFAKEFGLTVAPAPGQGRRALYLTGT(A/V/D)AAMQTAFGVTFATKIMEGTKYRVREGDICLPKELIGHV
DAVLGLDNRPQAKPHFRHHKPAATSVSYTPVQVGQLYGFPSGAKATGQTIGLIELGGGFRAADITAYFKTLGQTAPKVTAV
LVDKAKNTPTTS(S/K/G)SADGEVMLDIEVAAAVAPGANIAVYFAP(N/D)(T/S)(D/A/T/N)QGFIDAISQAVHDTV
NKPSVISISW(G/S)GPESTWTAQSLAALDAACQSAAALGITITVAAGD(D/S/N)G S(T/A)(D/N/G)GV(K/Q/D)G
TVNHV(D/S/H)FPASSPHVLGCGGTKLLGSGTTITSEVVWNELTANEGATGGGVSNVFPLPTWQAKSNVPKPTVAAGGRG
VPDVSGNADPSTGYTVRVDGSTFPIGGTSAVAPLWAGLIALCNAQNKTTAGFINPALYAAAAAKSFRDITSGNNGGFKAGP
GWDACTGLGSPIGTAIAKTLAPATKSTSKTAVKNAPEIRFRPHKKAPTKTAAKTPALRRLK;
Homologue 19
(SEQ ID NO: 87)
MPTSSRFASQSRVPLPGSERKPFVPAGAPKAAKTPKVSTAVKTVPATGRIRVSLIVPPKQPLDTKRLGKLDARLSRAQFAA
RHGADPASVRLVKAFAKEFGLTVEPITQPGRCTVQLSGT(C/V/D)AAMRKAFAISLVEHTTEQGKFRLREGEISLPAELE
GHVLAVLGLDNRPQAKPHFRIAKPRATNVSYTPVQVAQMYGFPAGATATGQTIGIIELGGGYRAADLTAYFKTLGLPAPTV
TAVPIDGGKNTPGNA(N/S/K/G)GADGEVMLDIEVCAAVAQGAKIAVYFTT(N/D)(T/S)(D/A/T/N)QGFIDAITTA
VHDSTNKPSVISISW(G//S)GPESSWTEQSMTALDAACQAAAAVGVTITVAAGD(N/S)G S(S/T/S)(D/N/G)GA
(S/Q/D)GDNV(D/S/H)FPASSPHVLACGGTKLVGSGSTITSEVVWDETSNDEGATGGGVSTVFALPTWQKNANVPSPTT
SAGGRGVPDVSGDADPSTGYTIRVDSETTVIGGTSAVAPLWAGLIALANAQNKVAAGFVNPALYAAGAKKAFRDITQGNNG
SFSAGPGWDACTGLGSPVGNLVIQAVAPKSTTTKKAKKGKTK;
and
Homologue 26
(SEQ ID NO: 88)
MHSYLKQQSHMQSYLEQENHMRSYLEMRKKPYFDDLANIRPGGLTPAQVCQAYQFAKVQPVRPVKLGIVSLAGQYLSSDMS
KAFTGYGLPTPVVSTAGSQVLGDLWSNVE(N/S/K/G)MMDIEIAGAAWAYATGTAATLLMQFEP(N/D)(N/T/S)(E/
D/A/T/N)TGIPNAINALVAAGCEVISISW(G/S)APANLQTMEAITARKEACKQAAVQNVHVFAASGD(E/S/N)SL(N/
T/A)(D/N/G)(G/Q/D)TNSRTP(D/S/H)DPCCDPNVWGVGGTRLVLQADGSIAQESAWGDGNAADKGGGGGFDSREPL
PDYQVGVVHSEHRGSPDSSANADPGTGYAIVANGQWLIGGGTSASAPLTAGYVAAILSTLPGPISQSVLQRKLYTAHKTAF
RDILLGSNGAPARPGWEEATGLGSINGPGLAAALQS.

The polypeptides disclosed herein are Kumamolisin homologue polypeptides and modified versions thereof that have been identified as having similar, improved, or complementary activity compared to Kumamolisin-As in hydrolyzing proline (P)- and glutamine (Q)-rich components of gluten known as ‘gliadins’ believed responsible for the bulk of the immune response in most celiac sprue patients. Numerous other Kumamolisin homologues tested by the inventors possessed little or no such gliadin hydrolyzing activity. Thus, the polypeptides disclosed herein can be used to treat celiac sprue. The amino acid sequences disclosed herein are for the preprocessed version of the polypeptides, which may hydrolyze their substrates in a processed form. Thus, use of the processed versions of the polypeptides are covered herein. As will be understood by those of skill in the art, the exact processing of the polypeptides may differ from one cell type or set of conditions to another. In one embodiment, the processed forms of the homologues are devoid of the residues shown in Table 1 below, which s a comparison of the residues of Kummamolisin and the homologues disclosed herein that are present in the pre-processed form but not in the processed form.

TABLE 1

| | Pre-Protein |
|---|---|
| Kumamolisin | 1-189 |
| Hom 1 | 1-148 |
| Hom 2 | 1-182 |
| Hom 4 | 1-174 |
| Hom 5 | 1-187 |
| Hom 6 | 1-157 |
| Hom 9 | 1-173 |
| Hom 10 | 1-135 |
| Hom 12 | 1-171 |
| Hom 13 | 1-172 |
| Hom 14 | 1-173 |
| Hom 15 | 1-169 |
| Hom 16 | 1-173 |
| Hom 17 | 1-178 |
| Hom 19 | 1-181 |
| Hom 26 | 1-42 |

In one embodiment, the one or more polypeptides are selected from the group consisting of the following, or processed versions thereof

Homologue 1 (NCBI YP_005536585)
(SEQ ID NO: 74)
MAPSDVEIVDPVAPEERITVTVLLRRRSSIPDQLIEGPDTLSRAELADRHGADPADVEAVRVAMSGAGLTVVGTDLPSRRV
TVAGTAEALMRTFGAELQIVRDASGFQHRARSGELRIPAALDGIVIAVLGLDNRPQAEARFRASQPEAARSFRPDALGRVY
RFPANTDGTGQTIAIVELGGGFRQSELDTYFGGLGIPAPQVLAVGVDGGQNLPSGDAGSADGEVLLDIEVAGALAPGARQV
VYFAPNTDRGFVDAVTTAVHADPTPAAVSISWGAPEDKWTAQARRAFDAALADAAALGVTVTAAAGDRGSADGEGGGGLHT
DFPASSPHLLACGGTKLAVADGGTVASETVWNGGERGGATGGGVSVAFGLPAYQRNAGVDKRRKTGKPGRGVPDVAAVADP
ATGYEVLVDGEQLVFGGTSAVAPLWAALVARLTQALGRPLGLLNTALYDGAQPGRTQPGFRDVTEGDNDISGKHGPYPARA
GWDACTGLGVPDGEALLAALRKPGKE;
Homologue 2 (NCBI ZP_04943175)
(SEQ ID NO: 97)
MQRGTKEGLNMARHLQADREPRIVPESKCLGQCDPAERIHVTIMLRRQEEGQLDALVHQLATGDARAKPVSRDAFAQRFSA
NPDDIRKTEDFAHRHQLTVDRVDPVESVVVLSGTIAQFEAAFSVKLERFEHRSIGQYRGRSGPIVLPDDIGDAVTAVLGLD
SRPQARPHFRFRPPFKPARGAAAVTFTPIQLASLYDFPAGDGAGQCIAIIELGGGYRAADIQQYFRGLGITTPPKLVDVNV
GTGRNAPTGEPNGPDGEVALDIEIAGAIAPAAKIAVYFAPNSDAGFIQAVNAAVTDKTNQPSVISISWGGPEAIWQAQSAQ
AFNRVLQAAAAQGITVCAASGDSGSGDGLQDGADHVDFPASSPYVLGCGGTQLDALPGQGIRSEVTWNDEASGGGAGGGGV
SALFDLPAWQQGLKVARADGTTTPLAKRGVPDVAGDASPQTGYEVSVAGTPAVMGGTSAVAPLWAALIARINAANGASAGW
INPVLYKUPGALRDITKGSNGTYAAASGWDACTGLGSPNGAQLATILARKPSS;
Homologue 4 (NCBI ZP_10028298)
(SEQ ID NO: 98)
MANHPLNGSERECLKDAQPIGKADPNERLEVTMLVRRRSHDAFEKHISALAAQGASAKHIDHDEFTKHFGADSADLAAVHA
FAQKHGLSVVESHEARRAVVLSGTVAQFDAAFGVSLQQYEHDGGTYRGRTGPIHLPDELNGVVDAVMGLDNRPQARPSFRT
RAQGNVRWTARAAGASTFTPVQLASLYDFPQGDGQNQCIGIIELGGGYRPADLKTYFASLNMKAPSVTAVSVDHGRNHPTG
DPNGPDGEVMLDIEVAGAVAPGAKIVVYFAPNTDAGFIDAIGTAIHDTKNKPSVISISWGGPESAWTQQAMNAFDQAFQSA
AALGVTICAASGDNGSGDGVGDGADHVDFPASSPYALGCGGTSLQASGNGIASETVWNDGANGGATGGGVSSFFALPAWQE
GLRVTRAGGAHSPLAMRGVPDVAGNADPVTGYEVRVDGHDMVIGGTSAVAPLWAGLIARINAIKGAPVGYINPHLYKDPLA
LVDITKGNNDDFHATAGWDACTGLGRPDGKKVKDAVS;
Homologue 5 (NCBI ZP_09078202)
(SEQ ID NO: 99)
AGRTSYTPLEVAALYNFPSIHCKDQCIGILELGGGYRPADLQTYFNGLGIPQPNITDVSVGGAANRPTGDPNGPDGEVVLD
IEVAAAVTPGAKIAVYFADNSDDGFLNAITTAIHDTRNKPSVISISWGKAEIGWTPQAINAMNQAFRDAAALGVTICCASG
DDGSTDRVQDGRYHVDFPASSPYVLACGGTRLESSGSTITQEVVWNEGALGGGATGGGVSDVFDRPNWQANANVPTSANPE
RRIGRGVPDWAGNADPATGYQILVDGTRAVIGGTSAVAPLFAGLIAIINQKLGHSVGFINPILYNLSAQHNVFHDITSGNN
DMSGQNGPYEAQPGWDACTGLGSPDGTKLMNAISEAHRLVSVG;
Homologue 6 (NCBI YP_003109679)
(SEQ ID NO: 77)
MAPEERRTLPGSAMPRPAGAQVLGQIPDDERVEVTVVLQPRAPLPEPGPTPMSRAELADLRSPPEGALEAIARYVAGQGLE
VIAADAPRRRIVLAGSAARIAALFGISFVRLQLEGRRYRTYEGEISLPAELAPLVVAVLGLDTRPFARSHRRPAVAPNAPT
TAPTVARAYDFPTAYDGRGTTIGFIELGGGFQESDLVRYCEGLGLSTPQVSVVGVDGARNAPTGDPNGPDAEVMLDLEVAT
GVANGADLVLYMAANTDAAFYSAIATALRDATHAPVAISISWGAPEESYPATTIAAFESVLEEAVHVGVTVLVAAGDQGST
DGVDDGRAHVDYPAASPYVLACGGTRLDLDGTTIVAETVWNDLPNGGATGGGISALFPVPSWQAGIAMPPSANPGAGPGRG
VPDVAGNADPDTGYRIVVDGVATVVGGTSAVAPLWAGLVARCHQAGARGGFWNPLLYAARGSSAFHEITVGSNGAYDAGPI
WNACCGLGSPNGTAILQTLRA;
Homologue #6 mutant:
(SEQ ID NO: 78)
MAPEERRTLPGSAMPRPAGAQVLGQIPDDERVEVTVVLQPRAPLPEPGPTPMSRAELADLRSPPEGALEAIARYVAGQGLE
VIAADAPRRRIVLAGSAARIAALFGISFVRLQLEGRRYRTYEGEISLPAELAPLVVAVLGLDTRPFARSHRRPAVAPNAPT
TAPTVARAYDFPTAYDGRGTTIGFIELGGGFQESDLVRYCEGLGLSTPQVSVVGVDGARNAPTGDPNGPDAEVMLDLEVAT
GVANGADLVLYMAANTDAAFYSAIATALRDATHAPVAISISWSAPEESYPATTIAAFESVLEEAVHVGVTVLVAAGDQGST
GGVDDGRAHVHYPAASPYVLACGGTRLDLDGTTIVAETVWNDLPNGGATGGGISALFPVPSWQAGIAMPPSANPGAGPGRG
VPDVAGNADPDTGYRIVVDGVATVVGGTSAVAPLWAGLVARCHQAGARGGFWNPLLYAARGSSAFHEITVGSNGAYDAGPI
WNACCGLGSPNGTAILQTLRA;
Homologue 9 (NCBI YP_005042475)
(SEQ ID NO: 102)
MTKQPVSGSSDKIHPDDAKCIGDCDPSEQIEVIVMLRRKDEAGFRQMMSRIDAGEAPGQAVSREEFDRRFTASDEDIDKVK
AFAKQYGLSVERAETETRSVVLKGTIEQFQKAFDVKLERFQHHNIGEYRGRTGPVNVPDEMHDAVTAVLGLDSKPQARPHF
RFRPPFKPLRGAAPASFSPVDLAKLYDFPDGDGAGQCIAIIELGGGYRDSDLSAYFSKLGVKAPTVVPVGVDGGKNAPTGN
PNGPDGEVTLDIEIAGAIAPGARIAVYFAPNSDAGFVDAVNRALHDAANKPSVISISWGGPESNWSPQSMSAFNDVLQSAA
ALGVTVCAASGDGGSADGVGDGADHVDFPASSPYVLGCGGTSLAASGAGIAKEVVWNDGDQGGAGGGGVSGTFALPVWQKG
LSVTRNGKHIALAKRGVPDVAGDASPQTGYEVLIDGEDTVVGGTSAVAPLWAALIARINAIDASPAGFVNPKLYKAKTAFR
DITEGNNGSFSAAAGWDACTGMGSPDGGKIAAALKPAKPSQSAGQQ;
Homologue 10 (NCBI YP_711059)
(SEQ ID NO: 103)
MGRLQGSYRPSLGTPVGPVPDDQPIDVTVVLRPTAADDFRADPDDVAAVRAFAGRAGLDVAEVDEPARTVRLRGPAAAART
AFDTPLALYDSGGRAIRGREGDLGLPDELDDRVVAVLGLDERPAARPRFQPAASARQGLTALQVARAYDFPAATGEGQTIA
IIELGGGFGQADLDTYFGGLDLPTPAVSAVGVQGAANVPGGDPDGADGEVLLDIEVAGAVAPGAAQVVYFAPNTDAGFLAA
INAAAAATPRPAAISISWGGPESSWTAQAMRAYDQAFAAARAAGITVLAAAGDAGADDATDRLVADFPAGSPNVIACGGTK
LTLDAAGARASEVVWNEAADSATGGGYSATFTRPAWQPAAVGRYRGLPDISGNADPQTGYRVVVDGQPTVVGGTSAVAPLL
AGLVARLAQLTGAPVADLAAVAYANPAAFTDITAGDNQGYPARSGWDPASGLGSPVGTKLLTAVGGPTPPPTTPPPTTPPP
TTPPPTIPPPTTPPTQTVDAADRALWSAVATWAGGTHTGANARAAKAVRAWAQAKSLA;
Homologue 12 (NCBI YP_003658449)
(SEQ ID NO: 104)
MTQPRYTPLPGSEREAPLLAARSNATAARASRAQTASATVVLRRRSELPEALVLDQQFISSDELAARYGADPVDIEKVRSV
LERFKVSVVEVDAASRRVKVEGAVADIERAFNIALHSASGTDPHSGRGFEYRYRTGVLSVPAELGGIVTAVLGLDNRRQAE
TRLRVVPAAALGSSYTPVQLGEIYNFPQDATGAGQRIAIIELGGGYTPAGLRRYFASLGVVPPKVAAVSVDGAQNAPGPDP
GADGEVQLDVEVAGALAPGAHVLVYFAPNTDQGFLDAVSQAAHATPPPTAISISWGASEDSWTASARDALNQALRDAAALG
VTVTAAAGDSGSSDGVPDRRAHVDFPASSPYVLATGGTSLRADPATGVVQSETVWNDSQGSTGGGVSDVFPRPAWQAHVDV
PHAGRGVPDVSAVADPATGYQVLVDNQPAVIGGTSAVAPLWAALVARLAESLGRPLGLLQPLVYPRTPGSTAYPGFRDITI
GNNGAYKAGKGWDAATGLGVPDGTELLAHLRGLNGSE;
Homologue 13 (NCBI YP_004348568)
(SEQ ID NO: 105)
MARHLHAGSEPKVITESKCIGACDPAERIHVTVMLRREGEQALDALVDKLASGDPAAKPVSREDFAKRFGARADDIQHTEA
FAKRHQLTVERVDPVQSVVELAGTIAQFENAFGVKLEKYEHHAIGSFRARTGAIALPDELHDAVTAVLGLDTRPQAHPHFR
FRPPFQPARSGAGTSYTPLQLASIYNFPEGDGAGQCIALVELGGGYRAADIRQYFEQLGVKPPKLVDVSVNGGRNAPTDDP
NGPDGEVALDIEVAGAIAPGATIAVYFAGNSDAGFIQSVNQAIHDSTNRPSVVSISWGGPEASWTQQSITAFNNVLKTAAS
LGVTVCAASGDSGSSDGLQDGSNHVDFPASSPYVLACGGTTLDAQAGQGIRREVVWNDEAASGGAGGGGVSAVFPAPSYQK
GLSAKATGGGSTPLSQRGVPDVAGDASPTTGYIISIAGTTAVLGGTSAVAPLWAALIARINANGKSPVGWANPKLYAQPGA
FHDITQGNNGAFAASEGWDACTGLGSPDGAKVAAALQGASGGSQQGRATGA;
Homologue 14 (NCBI YP_001861188)
(SEQ ID NO: 106)
HMTKHPLPGSERVLAPGSKVVAQCDPSETIEVVVVLRRKNEQQFAQMMKTIEAGAAGARPLTREELEQRFGALPEDIAKLK
AFAAQHGLSVVREDASARTVVLSGRIEQFQQAFDVQLQHYEHQSMGRFRGRTGAISVPDELHGVVTAVLGLDDRPQARPHF
RIRPPFQPARAQSASSFTPLQLASLYRFPQGDGSGQCIGIVELGGGYRTADLDSYFSSLGVGSPKVVAVGVDQSGNQPTGD
PNGPDGEVTLDIEIAGALAPAATIAVYFTTNSDAGFIDAVSQAVHDRTNQPSVISISWGAPESMWTAQSMKALNDVLQSAA
AIGVTVCAASGDSGSSDGVGDGRDHVDFPASSPYVLACGGTSLQGSGRTVAHEVVWNDGSNGGATGGGVSGAFPVPAWQEG
LSTSAAQGGQRALTGRGVPDVAGDASPLTGYDVIVDGNNTVIGGTSAVAPLWAALIARINGAKGAPVGFVNPKLYKASACN
DITQGNNGSYAATTGWDACTGLGSPDGVKVAAAL;
Homologue 15 (NCBI YP_002754884)
(SEQ ID NO: 107)
MSPIASRRSALPLSERPAPENARALAAVEPDRTMTVSVLVRRKKPLVLADLEGKKLTHREFERRYGASEKDFATIAKFAAG
HGLAVDHHASSLARRTVVLRGTARQMQQAFGVTLHDYEDSETQQRYHSFTGAITVPAAHARIIESVLGLDARPIAKPHFRV
RKRSAAATGAVSFNPPQVASLYSFPTGVDGSGETIGILELGGGYETSDIQQYFSGLGIQPPTVVAVSVDGAVNAPGNPNGA
DGEVALDIQVAGSIAPGAKLAVYFAPNTEQGFVDAITTAVHDTANKPSVLSISWGGPESSWPQAAAQSLNNACESAAALGV
TITVASGDNGSTDGVQDGQNHVDFPASSPYVLACGGTYLAAVNNGVPQESVWDDLASGGGATGGGVSALFPLPAWQTGANV
PGGSMRGVPDVAGDASPESGYNVLVDGQPQVVGGTSAVAPLWAALIALVNQQKGEAAGFVNAALYQNPSAFHDITQGSNGA
YAAAPGWDPCTGLGSPMGTAIAKILA;
Homologue 16 (NCBI YP_003387700)
(SEQ ID NO: 108)
MSAFDQLVPLPGSEKTVPDAAPSQTLDPNEVLTVTIRIRRKRTLASLVSTTAPVTEVVSRSEYASRFGADPAIVKQVEAFA
SAYDLSLVEQSLARRSVLLRGTVAQMEQAFGVSLANYQLADGTVFRGRTGVVNVPSELVEHIEGVFGLDNRPQARAHFQVY
KPEKGTKVAPRAGGISYTPPQLARLYNFPTGVTGKGQCIAIIELGGGFRTADIKTYFGGLGLKPPTVVAVSVDGGHNAPST
ADSADGEVMLDIDVAGGVAPGAKIVVYFAPNTDQGFLDAITTAMHDTKNKPSVISISWGAAESNWTPQALTSFNQAFQAAA
ALGITVCAAAGDTGSDDSVGDGKAHVDFPASSPFVLACGGTKLTATDNVIASEVVWHESKTSATGGGVSDVFDLPDYQQKS
HVPPSVNDKTRIGRGVPDVAAVADPVTGYAVRVDGSNLVFGGTSAVAPLMAGLIALINQQRGKAVGFIHPLIYANPSAFRD
ITQGNNTTTTGNKGYAATTGWDACTGLGVADGKKLASVLTATPVA;
Homologue 17 (NCBI YP_004216463)
(SEQ ID NO: 109)
MAATPRFASQPRVTLPGSQKHPLTTDTEVPPPAPVKAAATKLSATPFTVTVIVKRKNPLNLKQVLKPAGRLTHAAFAKAHG
PSPDGVKLVKAFAKEFGLTVAPAPGQGRRALYLTGTAAAMQTAFGVTFATKIMEGTKYRVREGDICLPKELIGHVDAVLGL
DNRPQAKPHFRHHKPAATSVSYTPVQVGQLYGFPSGAKATGQTIGLIELGGGFRAADITAYFKTLGQTAPKVTAVLVDKAK
NTPTTSSSADGEVMLDIEVAAAVAPGANIAVYFAPNTDQGFIDAISQAVHDTVNKPSVISISWGGPESTWTAQSLAALDAA
CQSAAALGITITVAAGDDGSTDGVKGTVNHVDFPASSPHVLGCGGTKLLGSGTTITSEVVWNELTANEGATGGGVSNVFPL
PTWQAKSNVPKPTVAAGGRGVPDVSGNADPSTGYTVRVDGSTFPIGGTSAVAPLWAGLIALCNAQNKTTAGFINPALYAAA
AAKSFRDITSGNNGGFKAGPGWDACTGLGSPIGTAIAKTLAPATKSTSKTAVKNAPEIRFRPHKKAPTKTAAKTPALRRL
K;
Homologue 19 (NCBI YP_005056054)
(SEQ ID NO: 110)
MPTSSRFASQSRVPLPGSERKPFVPAGAPKAAKTPKVSTAVKTVPATGRIRVSLIVPPKQPLDTKRLGKLDARLSRAQFAA
RHGADPASVRLVKAFAKEFGLTVEPITQPGRCTVQLSGTCAAMRKAFAISLVEHTTEQGKFRLREGEISLPAELEGHVLAV
LGLDNRPQAKPHFRIAKPRATNVSYTPVQVAQMYGFPAGATATGQTIGIIELGGGYRAADLTAYFKTLGLPAPTVTAVPID
GGKNTPGNANGADGEVMLDIEVCAAVAQGAKIAVYFTTNTDQGFIDAITTAVHDSTNKPSVISISWGGPESSWTEQSMTAL
DAACQAAAAVGVTITVAAGDNGSSDGASGDNVDFPASSPHVLACGGTKLVGSGSTITSEVVWDETSNDEGATGGGVSTVFA
LPTWQKNANVPSPTTSAGGRGVPDVSGDADPSTGYTIRVDSETTVIGGTSAVAPLWAGLIALANAQNKVAAGFVNPALYAA
GAKKAFRDITQGNNGSFSAGPGWDACTGLGSPVGNLVIQAVAPKSTTTKKAKKGKTK;
and
Homologue 26 (NCBI YP_004030750)
(SEQ ID NO: 111)
MHSYLKQQSHMQSYLEQENHMRSYLEMRKKPYFDDLANIRPGGLTPAQVCQAYQFAKVQPVRPVKLGIVSLAGQYLSSDMS
KAFTGYGLPTPVVSTAGSQVLGDLWSNVENMMDIEIAGAAWAYATGTAATLLMQFEPNNETGIPNAINALVAAGCEVISIS
WGAPANLQTMEAITARKEACKQAAVQNVHVFAASGDESLNDGTNSRTPDDPCCDPNVWGVGGTRLVLQADGSIAQESAWGD
GNAADKGGGGGFDSREPLPDYQVGVVHSEHRGSPDSSANADPGTGYAIVANGQWLIGGGTSASAPLTAGYVAAILSTLPGP
ISQSVLQRKLYTAHKTAFRDILLGSNGAPARPGWEEATGLGSINGPGLAAALQS.

In one embodiment, the one or more polypeptides comprise one or more polypeptides comprising or consisting of the amino acid sequence of a polypeptide selected from the group consisting of SEQ ID NOs: 74, 75, 77, 78, 82, 88, 98, 105, 111, or processed versions thereof. In a further embodiment, the one or more polypeptides comprise a polypeptide that comprises or consists of the amino acid sequence:

Homologue 4 mutant
(SEQ ID NO: 89)
MANHPLNGSERECLKDAQPIGKADPNERLEVTMLVRRRSHDAFEKHISAL
AAQGASAKHIDHDEFTKHFGADSADLAAVHAFAQKHGLSVVESHEARRAV
VLSGTVAQFDAAFGVSLQQYEHDGGTYRGRTGPIHLPDELNGVVDAVMGL
DNRPQARPSFRTRAQGNVRWTARAAGASTFTPVQLASLYDFPQGDGQNQC
IGIIELGGGYRPADLKTYFASLNMKAPSVTAVSVDHGRNHPTGDPNGPDG
EVMLDIEVAGAVAPGAKIVVYFAPNTDAGFIDAIGTAIHDTKNKPSVISI
SWSGPESAWTQQAMNAFDQAFQSAAALGVTICAASGDNGSGGGVGDGADH
VHFPASSPYALGCGGTSLQASGNGIASETVWNDGANGGATGGGVSSFFAL
PAWQEGLRVTRAGGAHSPLAMRGVPDVAGNADPVTGYEVRVDGHDMVIGG
TSAVAPLWAGLIARINAIKGAPVGYINPHLYKDPLALVDITKGNNDDFHA
TAGWDACTGLGRPDGKKVKDAVS,

or a processed version thereof.

The methods may comprise administration of the one or more polypeptides together with any other suitable active agent to treat celiac sprue. In various non-limiting embodiments, the methods further comprise administering to the subject an amount of one or more further polypeptides comprising an amino acid sequence selected from the group consisting of:
(A) an amino acid sequence at least 75%, 80%, 85%, 90%, 95%, or 100% identical to the amino acid sequence of SEQ ID NO:35, wherein
   (i) the poly-peptide degrades a PQPQLP (SEQ ID NO:34) peptide at pH 4; and
   (ii) residue 278 is Ser, residue 78 is Glu, and residue 82 is Asp
(B) an an amino acid sequence at least 75%, 80%, 85%, 90%, 95%, or 100% identical to the amino acid sequence of SEQ ID NO:1, wherein
   (i) the polypeptide degrades a PQPQLP (SEQ ID NO:34) peptide at pH 4; and
   (ii) residue 467 is Ser, residue 267 is Gln, and residue 271 is Asp.

The one or more further polypeptides have been disclosed for use in treating celiac sprue (see WO2013/023151). The further polypeptides are either the processed version of Kumamolisin-As (SEQ ID NO:67) or the preprocessed version of Kumamolisin-As (SEQ ID NO:33), or modified versions thereof, which are known as a member of the sedolisin family of serine-carboxyl peptidases, and utilizes the key catalytic triad Ser²⁷⁸-Glu⁷⁸-Asp⁸² in its processed form to hydrolyze its substrate (Ser⁴⁶⁷-Glu²⁶⁷-Asp²⁷¹ in the pre-processed form) Its maximal activity is at pH ˜4.0. While the native substrate for Kumamolisin-As is unknown, it has been previously shown to degrade collagen under acidic conditions. In addition, this enzyme has been shown to be thermostable, with an ideal temperature at 60° C., but still showing significant activity at 37° C.

The further polypeptides may comprise one or more amino acid changes from SEQ ID NO: 67 (wild type processed Kumamolisin-As) at one or more residues selected from the group consisting of residues 73, 102, 103, 104, 130, 165, 168, 169, 172, and 179 (numbering based on the wild type processed Kumamolisin-As amino acid sequence). In non-limiting embodiments, the one or more changes relative to the wild type processed Kumamolisin-As amino acid sequence (SEQ ID NO:67) may be selected from the group consisting of:

| Wild type Residue# | AA change |
|---|---|
| S73 | K, G |
| N102 | D |
| T103 | S |
| D104 | A, T, N |
| G130 | S |
| S165 | N |
| T168 | A |
| D169 | N, G |
| Q172 | D |
| D179 | S, H |

In various further non-limiting embodiments, the one or more changes relative to the wild type processed Kumamolisin-As amino acid sequence may include at least N102D. In another embodiment the one or more changes relative to the wild type Kumamolisin-As amino acid sequence may include at least N102D and D169N or D169G. In another embodiment the one or more changes relative to the wild type Kumamolisin-As amino acid sequence may include at least N102D, D169G, and D179H. In another embodiment the one or more changes relative to the wild type Kumamolisin-As amino acid sequence may include at least S73K, D104T, N102D, G130S, D169G, and D179H.

The further polypeptides may comprise one or more amino acid changes from SEQ ID NO: 33 (wild type pre-processed Kumamolisin-As) at one or more residues selected from the group consisting of residues 119, 262, 291, 292, 293, 319, 354, 357, 358, 361, and 368 (numbering based on the wild type pre-processed Kumamolisin-As amino acid sequence). In non-limiting embodiments, the one or more changes relative to the wild type Kumamolisin-As amino acid sequence may be selected from the group consisting of:

| Wild type Residue# | AA change |
|---|---|
| V119 | D |
| S262 | K, G |
| N291 | D |
| T292 | S |
| D293 | A, T, N |
| G319 | S |
| S354 | N |
| T357 | A |
| D358 | N, G |
| Q361 | D |
| D368 | S, H |

In various further non-limiting embodiments, the one or more changes relative to the wild type Kumamolisin-As amino acid sequence may include at least N291D. In another embodiment the one or more changes relative to the wild type Kumamolisin-As amino acid sequence may include at least N291D and 358N or 358G. In another embodiment the one or more changes relative to the wild type Kumamolisin-As amino acid sequence may include at least N291D, 358G, and 368H. In another embodiment the one or more changes relative to the wild type Kumamolisin-As amino acid sequence may include at least V119D, S262K, D293T, N291D, G319S, D358G, and D368H.

As used herein, “at least 75% identical” means that the polypeptide differs in its full length amino acid sequence by 25% or less (including any amino acid substitutions, deletions, additions, or insertions) from the polypeptide defined by SEQ ID NO:1 or 35.

In various further embodiments, the one or more further polypeptides comprise or consist of an amino acid sequence at least 75% identical to any one of SEQ ID NOS:2-33 or 36-67, or, alternatively, 2-32 or 36-66. The polypeptides represented by these SEQ ID NOS are specific examples of polypeptides with improved protease activity at pH 4 against the oligopeptide PQPQLP (SEQ ID NO: 34) (a substrate representative of gliadin) compared to wild type Kumamolisin-As. In various preferred embodiment, the one or more further polypeptides comprise or consist of an amino acid sequence at least 76%, 77%, 78%, 79%, 80%, 81%, 82%, 83%, 84%, 85%, 86%, 87%, 88%, 89%, 90%, 91%, 92%, 92%, 93%, 94%, 95%, 96%, 97%, 98%, or 99% identical to an amino acid sequence according to any one of SEQ ID NOS:36-66. In a further embodiment the one or more polypeptides comprise or consist of an amino acid sequence according to any one of SEQ ID NOS: 2-33 or 36-67 or, alternatively, 2-32 or 36-66.

In one embodiment, the one or more further polypeptide comprises or consists of a polypeptide comprising the amino acid sequence shown below (KumaMax™):

(SEQ ID NO: 90)
MSDMEKPWKEGEEARAVLQGHARAQAPQAVDKGPVAGDERMAVTVVLRRQ
RAGELAAHVERQAAIAPHAREHLKREAFAASHGASLDDFAELRRFADAHG
LALDRANVAAGTAVLSGPDDAINRAFGVELRHFDHPDGSYRSYLGEVTVP
ASIAPMIEAVLGLDTRPVARPHFRMQRRAEGGFEARSQAAAPTAYTPLDV
AQAYQFPEGLDGQGQCIAIIELGGGYDEASLAQYFASLGVPAPQVVSVSV
DGASNQPTGDPKGPDGEVELDIEVAGALAPGAKFAVYFAPDTTAGFLDAI
TTAIHDPTLKPSVVSISWSGPEDSWTSAAIAAMNRAFLDAAALGVTVLAA
AGDSGSTGGEQDGLYHVHFPAASPYVLACGGTRLVASGGRIAQETVWNDG
PDGGATGGGVSRIFPLPAWQEHANVPPSANPGASSGRGVPDLAGNADPAT
GYEVVIDGEATVIGGTSAVAPLFAALVARINQKLGKAVGYLNPTLYQLPA
DVFHDITEGNNDIANRAQIYQAGPGWDPCTGLGSPIGVRLLQALLPSASQ
PQP,

or a processed version thereof.

In one embodiment, the method comprises administering Homologue 4 or full length mutant Homologue 4 (SEQ ID NOs: 75, 89, and/or 98), or processed versions thereof, together with the one or more of the further polypeptides disclosed herein, including but not limited to KumaMa™ (SEQ ID NO: 90), or a processed version thereof. As shown in the examples that follow, Homologue 4 (SEQ ID NO: 98) has increased activity against γ-gliadin peptide (amino acid sequence IQPQQPAQL (SEQ ID NO: 92)) compared to Kumamolisin polypeptides. Thus, administering a combination of Homologue 4 (SEQ ID NO: 98) or a processed version thereof and one or more Kumamolisin polypeptides (such as KumaMax™ (SEQ ID NO: 90), or a processed version thereof) may provide an improved therapy for gluten digestion. In a further embodiment, the Homologue 4 polypeptide comprises or consists of the full length Hom 4 mutant (SEQ ID NO: 89) or a processed version thereof, which is shown in the examples below to provide significantly improved activity against degradation products of gluten in the stomach that have been specifically linked to celiac disease: the 33mer peptide (LQLQPFPQPQLPYPQPQLPYPQPQLPYPQRQPF (SEQ ID NO: 72)) and the 26mer peptide (FLQPQQPFPQQPQQPYPQQPQQPFPQ (SEQ ID NO: 73)).

In another embodiment, the method comprises administering Homologue 1 (SEQ ID NO: 74), Homologue 6 (SEQ ID NO: 77), and/or the Homologue 6 mutant (SEQ ID NO: 78), or processed versions thereof, together with the one or more of the further polypeptides disclosed herein, including but not limited to KumaMax™ (SEQ ID NO: 90), or a processed version thereof. As demonstrated in the examples that follow, Homologue 1 (SEQ ID NO: 74) is optimally active through pH 5, and the Homologue 6 mutant demonstrates optimal activity at a pH level below that of the other homologues and the further Kumamolisin related polypeptides. As a result, Homologue 1 (SEQ ID NO: 74), Homologue 6 (SEQ ID NO: 77), and/or the Homologue 6 mutant (SEQ ID NO: 78), or processed versions thereof can be used alone in appropriate pH environments, or used in combination with the one or more further polypeptides to expand the pH profile of the one or more further polypeptides, to for example, more accurately mimic the pH of the stomach.

In a further embodiment, the method comprises administering Homologue 26 (SEQ ID NO: 88 or 111) or a processed version thereof, together with the one or more further polypeptides, including but not limited to KumaMax™ (SEQ ID NO: 90), or a processed version thereof. As shown in the examples that follow, Homologue 26 (SEQ ID NO:111) has very strong activity in breaking down the 33mer gliadin peptide, and thus can be used for treating celiac sprue disease, either alone or on combination with the one or more further polypeptides, including but not limited to KumaMax™ (SEQ ID NO: 90).

Celiac sprue (also known as celiac disease or gluten intolerance) is a highly prevalent disease in which dietary proteins found in wheat, barley, and rye products known as ‘glutens’ evoke an immune response in the small intestine of genetically predisposed individuals. The resulting inflammation can lead to the degradation of the villi of the small intestine, impeding the absorption of nutrients. Symptoms can appear in early childhood or later in life, and range widely in severity, from diarrhea, fatigue, weight loss, abdominal pain, bloating, excessive gas, indigestion, constipation, abdominal distension, nausea/vomiting, anemia, bruising easily, depression, anxiety, growth delay in children, hair loss, dermatitis, missed menstrual periods, mouth ulcers, muscle cramps, joint pain, nosebleeds, seizures, tingling or numbness in hands or feet, delayed puberty, defects in tooth enamel, and neurological symptoms such as ataxia or paresthesia. There are currently no effective therapies for this lifelong disease except the total elimination of glutens from the diet. Although celiac sprue remains largely underdiagnosed, its' prevalence in the US and Europe is estimated at 0.5-1.0% of the population.

As used herein, “treating celiac sprue” means accomplishing one or more of the following: (a) reducing the severity of celiac sprue; (b) limiting or preventing development of symptoms characteristic of celiac sprue; (c) inhibiting worsening of symptoms characteristic of celiac sprue; (d) limiting or preventing recurrence of celiac sprue in patients that have previously had the disorder; (e) limiting or preventing recurrence of symptoms in patients that were previously symptomatic for celiac sprue; and (f) limiting development of celiac sprue in a subject at risk of developing celiac sprue, or not yet showing the clinical effects of celiac sprue.

The subject to be treated according to the methods of the invention may be any subject suffering from celiac sprue, including human subjects. The subject may be one already suffering from symptoms or one who is asymptomatic.

In one embodiment, the subject may have an HLA-DQ2 serotype; in another embodiment, the subject may have an HLA-DQA serotype. Polypeptides with increased activity against γ-gliadin (Homologues 1, 4, 5, and 9 (SEQ ID NOs: 74, 75, 76, 79, 89, 98, 99, and 102)) may be particularly useful for treating subjects with an HLA-DQ8 serotype. Polypeptides with increased activity against α2-gliadin and α9-gliadin and/or the 33-mer and 26-mer degradation products of gluten described herein (Homologues 4 mutant (SEQ ID NO: 89) and Homologues 13 and 26 (SEQ ID NOs: 82, 88, 105, and 111)) may be particularly useful for treating subjects with an HLA-DQ2 serotype.

As used herein, an “amount effective” refers to an amount of the polypeptide that is effective for treating celiac sprue. The polypeptides are typically formulated as a pharmaceutical composition, such as those disclosed above, and can be administered via any suitable route, including orally, parentally, by inhalation spray, or topically in dosage unit formulations containing conventional pharmaceutically acceptable carriers, adjuvants, and vehicles. In a preferred embodiment, the pharmaceutical compositions and formulations are orally administered, such as by tablets, pills, lozenges, elixirs, suspensions, emulsions, solutions, or syrups.

Dosage regimens can be adjusted to provide the optimum desired response (e.g., a therapeutic or prophylactic response). A suitable dosage range may, for instance, be 0.1 ug/kg-100 mg/kg body weight; alternatively, it may be 0.5 ug/kg to 50 mg/kg; 1 ug/kg to 25 mg/kg, or 5 ug/kg to 10 mg/kg body weight. The polypeptides can be delivered in a single bolus, or may be administered more than once (e.g., 2, 3, 4, 5, or more times) as determined by an attending physician.

In another aspect, the present invention provides isolated polypeptides selected from the group consisting of the following polypeptides, or processed versions thereof.

Homologue 2
(SEQ ID NO: 95)
MQRGTKEGLNMARHLQADREPRIVPESKCLGQCDPAERIHVTIMLRRQE
EGQLDALVHQLATGDARAKPVSRDAFAQRFSANPDDIRKTEDFAHRHQL
TVDRVDPVESVVVLSGT(I/V/D)AQFEAAFSVKLERFEHRSIGQYRGR
SGPIVLPDDIGDAVTAVLGLDSRPQARPHFRFRPPFKPARGAAAVTFTP
IQLASLYDFPAGDGAGQCIAIIELGGGYRAADIQQYFRGLGITTPPKLV
DVNVGTGRNAPTGEP(N/S/K/G)GPDGEVALDIEIAGAIAPAAKIAVY
FAP(N/D)(S/T)(D/A/T/N)AGFIQAVNAAVTDKTNQPSVISISW
(G/S)GPEAIWQAQSAQAFNRVLQAAAAQGITVCAASGD(S/N)GS(G/
T/A)(D/N/G)GL(Q/D)DGADHV(D/S/H)FPASSPYVLGCGGTQLDA
LPGQGIRSEVTWNDEASGGGAGGGGVSALFDLPAWQQGLKVARADGTTT
PLAKRGVPDVAGDASPQTGYEVSVAGTPAVMGGTSAVAPLWAALIARIN
AANGASAGWINPVLYKHPGALRDITKGSNGTYAAASGWDACTGLGSPNG
AQLATILARKPSS,

wherein one, two, three, four, five, six, seven, eight, nine, ten, or all eleven of the following are true: (i) AA residue 116 is V or D; (ii) AA residue 255 is S, K, or G; (iii) AA residue 284 is D; (iv) AA residue 285 is T; (v) AA residue 286 is A, T, or N; (vi) AA residue 312 is S; (vii) AA residue 347 is N; (viii) AA residue 350 is T or A; (ix) AA residue 351 is N or G; (x) AA residue 354 is D; and (xi) AA residue 361 is S or H;

Homologue 4
(SEQ ID NO: 75)
MANHPLNGSERECLKDAQPIGKADPNERLEVTMLVRRRSHDAFEKHISAL
AAQGASAKHIDHDEFTKHFGADSADLAAVHAFAQKHGLSVVESHEARRAV
VLSGT(V/D)AQFDAAFGVSLQQYEHDGGTYRGRTGPIHLPDELNGVVDA
VMGLDNRPQARPSFRTRAQGNVRWTARAAGASTFTPVQLASLYDFPQGDG
QNQCIGIIELGGGYRPADLKTYFASLNMKAPSVTAVSVDHGRNHPTGDP
(N/S/K/G)GPDGEVMLDIEVAGAVAPGAKIVVYFAP(N/D)(T/S)(D/
A/T/N)AGFIDAIGTAIHDTKNKPSVISISW(G/S)GPESAWTQQAMNAF
DQAFQSAAALGVTICAASGD(N/S)GS(G/T/A)(D/N/G)GV(G/Q/D)
DGADHV(D/S/H)FPASSPYALGCGGTSLQASGNGIASETVWNDGANGGA
TGGGVSSFFALPAWQEGLRVTRAGGAHSPLAMRGVPDVAGNADPVTGYEV
RVDGHDMVIGGTSAVAPLWAGLIARINAIKGAPVGYINPHLYKDPLALVD
ITKGNNDDFHATAGWDACTGLGRPDGKKVKDAVS;

wherein one, two, three, four, five, six, seven, eight, nine, ten, or all eleven of the following are true: (i) AA residue 106 is D; (ii) AA residue 246 is S, K, or G; (iii) AA residue 275 is D; (iv) AA residue 276 is S; (v) AA residue 277 is A, T, or N; (vi) AA residue 303 is S; (vii) AA residue 338 is S; (viii) AA residue 341 is T or A; (ix) AA residue 342 is N or G; (x) AA residue 345 is Q or D; and (xi) AA residue 352 is S or H;

Homologue 5
(SEQ ID NO: 76)
MNHDHSPTGGELSNWVRVPGSERAAVQGSRKVGPADPNEQMSVTVVVRRP
AADTAVTSMIEKVGAQPLSERRHLTREEFASTHGANPADLSKVEKFAHEH
NLQVKEVNAAAGTMVLSGT(V/D)TSFSKAFGVELSTYEHPDFTYRGRIG
HVHIPDYLADTIQSVLGLDNRPQASPRFRVLKEEGGVTTAHAGRTSYTPL
EVAALYNFPSIHCKDQCIGILELGGGYRPADLQTYFNGLGIPQPNITDVS
VGGAANRPTGDP(N/S/K/G)GPDGEVVLDIEVAAAVTPGAKIAVYFAD
(N/D)(S/T)(D/A/T/N)DGFLNAITTAIHDTRNKPSVISISW(G/S)K
AEIGWTPQAINAMNQAFRDAAALGVTICCASGD(D/S/N)GS(T/A)
(D//N/G)RV(Q/D)DGRYHV(D/S/H)FPASSPYVLACGGTRLESSGST
ITQEVVWNEGALGGGATGGGVSDVFDRPNWQANANVPTSANPERRIGRGV
PDWAGNADPATGYQILVDGTRAVIGGTSAVAPLFAGLIAIINQKLGHSVG
FINPILYNLSAQHNVFHDITSGNNDMSGQNGPYEAQPGWDACTGLGSPDG
TKLMNAISEAHRLVSVG;

wherein one, two, three, four, five, six, seven, eight, nine, ten, or all eleven of the following are true: (i) AA residue 120 is D; (ii) AA residue 259 is S, K, or G; (iii) AA residue 288 is D; (iv) AA residue 289 is T; (v) AA residue 290 is A, T, or N; (vi) AA residue 316 is S; (vii) AA residue 351 is S or N; (viii) AA residue 354 is A; (ix) AA residue 355 is N or G; (x) AA residue 358 is D; and (xi) AA residue 365 is S or H;

Homologue #6 mutant:
(SEQ ID NO: 78)
MAPEERRTLPGSAMPRPAGAQVLGQIPDDERVEVTVVLQPRAPLPEPGPT
PMSRAELADLRSPPEGALEAIARYVAGQGLEVIAADAPRRRIVLAGSAAR
IAALFGISFVRLQLEGRRYRTYEGEISLPAELAPLVVAVLGLDTRPFARS
HRRPAVAPNAPTTAPTVARAYDFPTAYDGRGTTIGFIELGGGFQESDLVR
YCEGLGLSTPQVSVVGVDGARNAPTGDPNGPDAEVMLDLEVATGVANGAD
LVLYMAANTDAAFYSAIATALRDATHAPVAISISWSAPEESYPATTIAAF
ESVLEEAVHVGVTVLVAAGDQGSTGGVDDGRAHVHYPAASPYVLACGGTR
LDLDGTTIVAETVWNDLPNGGATGGGISALFPVPSWQAGIAMPPSANPGA
GPGRGVPDVAGNADPDTGYRIVVDGVATVVGGTSAVAPLWAGLVARCHQA
GARGGFWNPLLYAARGSSAFHEITVGSNGAYDAGPIWNACCGLGSPNGTA
ILQTLRA;
Homologue 9
(SEQ ID NO: 79)
MTKQPVSGSSDKIHPDDAKCIGDCDPSEQIEVIVMLRRKDEAGFRQMMSR
IDAGEAPGQAVSREEFDRRFTASDEDIDKVKAFAKQYGLSVERAETETRS
VVLKGT(I/V/D)EQFQKAFDVKLERFQHHNIGEYRGRTGPVNVPDEMHD
AVTAVLGLDSKPQARPHFRFRPPFKPLRGAAPASFSPVDLAKLYDFPDGD
GAGQCIAIIELGGGYRDSDLSAYFSKLGVKAPTVVPVGVDGGKNAPTGNP
(N/S/K/G)GPDGEVTLDIEIAGAIAPGARIAVYFAP(N/D)(S/T)(D/
A/T/N)AGFVDAVNRALHDAANKPSVISISW(G/S)GPESNWSPQSMSAF
NDVLQSAAALGVTVCAASGD(G/S/N)GS(A/T)(D/N/G)GV(G/Q/D)
DGADHV(D/S/H)FPASSPYVLGCGGTSLAASGAGIAKEVVWNDGDQGGA
GGGGVSGTFALPVWQKGLSVTRNGKHIALAKRGVPDVAGDASPQTGYEVL
IDGEDTVVGGTSAVAPLWAALIARINAIDASPAGFVNPKLYKAKTAFRDI
TEGNNGSFSAAAGWDACTGMGSPDGGKIAAALKPAKPSQSAGQQ,

wherein one, two, three, four, five, six, seven, eight, nine, ten, or all eleven of the following are true: (i) AA residue 107 is V or D; (ii) AA residue 245 is S, K, or G; (iii) AA residue 274 is D; (iv) AA residue 275 is T; (v) AA residue 276 is A, T, or N; (vi) AA residue 302 is S; (vii) AA residue 337 is S or N; (viii) AA residue 340 is T or A; (ix) AA residue 341 is N or G; (x) AA residue 344 is Q or D; and (xi) AA residue 351 is S or H;

Homologue 10
(SEQ ID NO: 80)
MGRLQGSYRPSLGTPVGPVPDDQPIDVTVVLRPTAADDFRADPDDVAAVR
AFAGRAGLDVAEVDEPARTVRLRGP(A/V/D)AAARTAFDTPLALYDSGG
RAIRGREGDLGLPDELDDRVVAVLGLDERPAARPRFQPAASARQGLTALQ
VARAYDFPAATGEGQTIAIIELGGGFGQADLDTYFGGLDLPTPAVSAVGV
QGAANVPGGDP(/S/K/G)DGADGEVLLDIEVAGAVAPGAAQVVYFAP
(N/D)(T/S)(D/A/T/N)AGFLAAINAAAAATPRPAAISISWG(G/S)P
ESSWTAQAMRAYDQAFAAARAAGITVLAAAGD(A/S/N)GA(D/T/A)
(D/S/N/G)(A/Q/D)TDRLVA(D/S/H)FPAGSPNVIACGGTKLTLDAA
GARASEVVWNEAADSATGGGYSATFTRPAWQPAAVGRYRGLPDISGNADP
QTGYRVVVDGQPTVVGGTSAVAPLLAGLVARLAQLTGAPVADLAAVAYAN
PAAFTDITAGDNQGYPARSGWDPASGLGSPVGTKLLTAVGGPTPPPTTPP
PTTPPPTTPPPTIPPPTTPPTQTVDAADRALWSAVATWAGGTHTGANARA
AKAVRAWAQAKSLA,

wherein one, two, three, four, five, six, seven, eight, nine, ten, or all eleven of the following are true: (i) AA residue 76 is V or D; (ii) AA residue 206 is S, K, or G; (iii) AA residue 235 is D; (iv) AA residue 236 is S; (v) AA residue 237 is A, T, or N; (vi) AA residue 262 is S; (vii) AA residue 297 is S or N; (viii) AA residue 300 is T or A; (ix) AA residue 301 is N or G; (x) AA residue 302 is Q or D; and (xi) AA residue 309 is S or H;

Homologue 12
(SEQ ID NO: 81)
MTQPRYTPLPGSEREAPLLAARSNATAARASRAQTASATVVLRRRSELPE
ALVLDQQFISSDELAARYGADPVDIEKVRSVLERFKVSVVEVDAASRRVK
VEGA(V/D)ADIERAFNIALHSASGTDPHSGRGFEYRYRTGVLSVPAELG
GIVTAVLGLDNRRQAETRLRVVPAAALGSSYTPVQLGEIYNFPQDATGAG
QRIAIIELGGGYTPAGLRRYFASLGVVPPKVAAVSVDGAQNAPGPDP(G/
S/K/G)ADGEVQLDVEVAGALAPGAHVLVYFAP(N/D)(T/S)(D/A/T/
N)QGFLDAVSQAAHATPPPTAISISW(G/S)ASEDSWTASARDALNQALR
DAAALGVTVTAAAGD(S/N)GS(S/T/A)(D/N/G)GV(P/Q/D)DRRAH
V(D/S/H)FPASSPYVLATGGTSLRADPATGVVQSETVWNDSQGSTGGGV
SDVFPRPAWQAHVDVPHAGRGVPDVSAVADPATGYQVLVDNQPAVIGGTS
AVAPLWAALVARLAESLGRPLGLLQPLVYPRTPGSTAYPGFRDITIGNNG
AYKAGKGWDAATGLGVPDGTELLAHLRGLNGSE,

wherein one, two, three, four, five, six, seven, eight, nine, ten, or all eleven of the following are true: (i) AA residue 105 is D; (ii) AA residue 244 is S or K; (iii) AA residue 272 is D; (iv) AA residue 273 is S; (v) AA residue 274 is A, T, or N; (vi) AA residue 299 is S; (vii) AA residue 334 is N; (viii) AA residue 337 is T or A; (ix) AA residue 338 is N or G; (x) AA residue 341 is Q or D; and (xi) AA residue 348 is S or H;

Homologue 13
(SEQ ID NO: 82)
MARHLHAGSEPKVITESKCIGACDPAERIHVTVMLRREGEQALDALVDKL
ASGDPAAKPVSREDFAKRFGARADDIQHTEAFAKRHQLTVERVDPVQSVV
ELAGT(I/V/D)AQFENAFGVKLEKYEHHAIGSFRARTGAIALPDELHDA
VTAVLGLDTRPQAHPHFRFRPPFQPARSGAGTSYTPLQLASIYNFPEGDG
AGQCIALVELGGGYRAADIRQYFEQLGVKPPKLVDVSVNGGRNAPTDDP
(N/S/K/G)GPDGEVALDIEVAGAIAPGATIAVYFAG(N/D)(S/T)(D/
A/T/N)AGFIQSVNQAIHDSTNRPSVVSISW(G/S)GPEASWTQQSITAF
NNVLKTAASLGVTVCAASGD(S//N)GS(S/T/A)(D/N/G)GL(Q/D)D
GSNHV(D/S/H)FPASSPYVLACGGTTLDAQAGQGIRREVVWNDEAASGG
AGGGGVSAVFPAPSYQKGLSAKATGGGSTPLSQRGVPDVAGDASPTTGYI
ISIAGTTAVLGGTSAVAPLWAALIARINANGKSPVGWANPKLYAQPGAFH
DITQGNNGAFAASEGWDACTGLGSPDGAKVAAALQGASGGSQQGRATGA;

wherein one, two, three, four, five, six, seven, eight, nine, ten, or all eleven of the following are true: (i) AA residue 106 is V or D; (ii) AA residue 244 is S, K, or G; (iii) AA residue 273 is D; (iv) AA residue 274 is T; (v) AA residue 275 is A, T, or N; (vi) AA residue 301 is S; (vii) AA residue 336 is N; (viii) AA residue 339 is T or A; (ix) AA residue 340 is N or G; (x) AA residue 343 is D; and (viii) AA residue 350 is S or H;

Homologue 14
(SEQ ID NO: 83)
MTKHPLPGSERVLAPGSKVVAQCDPSETIEVVVVLRRKNEQQFAQMMKTI
EAGAAGARPLTREELEQRFGALPEDIAKLKAFAAQHGLSVVREDASARTV
VLSGR(I/V/D)EQFQQAFDVQLQHYEHQSMGRFRGRTGAISVPDELHGV
VTAVLGLDDRPQARPHFRIRPPFQPARAQSASSFTPLQLASLYRFPQGDG
SGQCIGIVELGGGYRTADLDSYFSSLGVGSPKVVAVGVDQSGNQPTGDP
(N/S/K/G)GPDGEVTLDIEIAGALAPAATIAVYFTT(N/D)(S/T)(D/
A/T/N)AGFIDAVSQAVHDRTNQPSVISISW(G/S)APESMWTAQSMKAL
NDVLQSAAAIGVTVCAASGD(S/N)GS(S/T/A)(D/N/G)GV(G/Q/D)
DGRDHV(D/S/H)FPASSPYVLACGGTSLQGSGRTVAHEVVWNDGSNGGA
TGGGVSGAFPVPAWQEGLSTSAAQGGQRALTGRGVPDVAGDASPLTGYDV
IVDGNNTVIGGTSAVAPLWAALIARINGAKGAPVGFVNPKLYKASACNDI
TQGNNGSYAATTGWDACTGLGSPDGVKVAAAL,

wherein one, two, three, four, five, six, seven, eight, nine, ten, or all eleven of the following are true: (i) AA residue 107 is V or D; (ii) AA residue 245 is S, K, or G; (iii) AA residue 274 is D; (iv) AA residue 275 is T; (v) AA residue 276 is A, T, or N; (vi) AA residue 302 is S; (vii) AA residue 337 is N; (viii) AA residue 340 is T or A; (ix) AA residue 341 is N or G; (x) AA residue 344 is Q or D; and (xi) AA residue 351 is S or H;

Homologue 15
(SEQ ID NO: 84)
MSPIASRRSALPLSERPAPENARALAAVEPDRTMTVSVLVRRKKPLVLAD
LEGKKLTHREFERRYGASEKDFATIAKFAAGHGLAVDHHASSLARRTVVL
RGT(A/V/D)RQMQQAFGVTLHDYEDSETQQRYHSFTGAITVPAAHARII
ESVLGLDARPIAKPHFRVRKRSAAATGAVSFNPPQVASLYSFPTGVDGSG
ETIGILELGGGYETSDIQQYFSGLGIQPPTVVAVSVDGAVNAPGNP(N/
S/K/G)GADGEVALDIQVAGSIAPGAKLAVYFAP(N/D)(T/S)(E/D/
A/T/N)QGFVDAITTAVHDTANKPSVLSISW(G/S)GPESSWPQAAAQSL
NNACESAAALGVTITVASGD(N/S)GS(T/A)(D/N/G)GV(Q/D)DGQN
HV(D/S/H)FPASSPYVLACGGTYLAAVNNGVPQESVWDDLASGGGATGG
GVSALFPLPAWQTGANVPGGSMRGVPDVAGDASPESGYNVLVDGQPQVVG
GTSAVAPLWAALIALVNQQKGEAAGFVNAALYQNPSAFHDITQGSNGAYA
AAPGWDPCTGLGSPMGTAIAKILA,

wherein one, two, three, four, five, six, seven, eight, nine, ten, or all eleven of the following are true: (i) AA residue 104 is V or D; (ii) AA residue 241 is S, K, or G; (iii) AA residue 270 is D; (iv) AA residue 271 is S; (v) AA residue 272 is D, A, T, or N; (vi) AA residue 398 is S; (vii) AA residue 33 is S; (viii) AA residue 336 is A; (ix) AA residue 337 is N or G; (x) AA residue 340 is D; and (xi) AA residue 347 is S or H;

Homologue 16
(SEQ ID NO: 85)
MSAFDQLVPLPGSEKTVPDAAPSQTLDPNEVLTVTIRIRRKRTLASLVST
TAPVTEVVSRSEYASRFGADPAIVKQVEAFASAYDLSLVEQSLARRSVLL
RGT(V/D)AQMEQAFGVSLANYQLADGTVFRGRTGVVNVPSELVEHIEGV
FGLDNRPQARAHFQVYKPEKGTKVAPRAGGISYTPPQLARLYNFPTGVTG
KGQCIAIIELGGGFRTADIKTYFGGLGLKPPTVVAVSVDGGHNAPSTA
(D/S/K/G)SADGEVMLDIDVAGGVAPGAKIVVYFAP(N/D)(T/S)(D/
A/T/N)QGFLDAITTAMHDTKNKPSVISISW(G/S)AAESNWTPQALTSF
NQAFQAAAALGITVCAAAGD(T/S/N)GS(D/T/A)(D/N/G)SV(G/Q/
D)DGKAHV(D/S/H)FPASSPFVLACGGTKLTATDNVIASEVVWHESKTS
ATGGGVSDVFDLPDYQQKSHVPPSVNDKTRIGRGVPDVAAVADPVTGYAV
RVDGSNLVFGGTSAVAPLMAGLIALINQQRGKAVGFIHPLIYANPSAFRD
ITQGNNTTTTGNKGYAATTGWDACTGLGVADGKKLASVLTATPVA,

wherein one, two, three, four, five, six, seven, eight, nine, ten, or all eleven of the following are true: (i) AA residue 104 is D; (ii) AA residue 245 is S, K, or G; (iii) AA residue 274 is D; (iv) AA residue 275 is S; (v) AA residue 276 is A, T, or N; (vi) AA residue 302 is S; (vii) AA residue 337 is S or N; (viii) AA residue 340 is T or A; (ix) AA residue 341 is N or G; (x) AA residue 344 is Q or D; and (xi) AA residue 351 is S or H;

Homologue 17
(SEQ ID NO: 86)
MAATPRFASQPRVTLPGSQKHPLTTDTEVPPPAPVKAAATKLSATPFTVT
VIVKRKNPLNLKQVLKPAGRLTHAAFAKAHGPSPDGVKLVKAFAKEFGLT
VAPAPGQGRRALYLTGT(A/V/D)AAMQTAFGVTFATKIMEGTKYRVREG
DICLPKELIGHVDAVLGLDNRPQAKPHFRHHKPAATSVSYTPVQVGQLYG
FPSGAKATGQTIGLIELGGGFRAADITAYFKTLGQTAPKVTAVLVDKAKN
TPTTS(S/K/G)SADGEVMLDIEVAAAVAPGANIAVYFAP(N/D)(T/S)
(D/A/T/N)QGFIDAISQAVHDTVNKPSVISISW(G/S)GPESTWTAQSL
AALDAACQSAAALGITITVAAGD(D/S/N)GS(T/A)(D/N/G)GV(K/
Q/D)GTVNHV(D/S/H)FPASSPHVLGCGGTKLLGSGTTITSEVVWNELT
ANEGATGGGVSNVFPLPTWQAKSNVPKPTVAAGGRGVPDVSGNADPSTGY
TVRVDGSTFPIGGTSAVAPLWAGLIALCNAQNKTTAGFINPALYAAAAAK
SFRDITSGNNGGFKAGPGWDACTGLGSPIGTAIAKTLAPATKSTSKTAVK
NAPEIRFRPHKKAPTKTAAKTPALRRLK,

wherein one, two, three, four, five, six, seven, eight, nine, ten, or all eleven of the following are true: (i) AA residue 118 is V or D; (ii) AA residue 250 is K, or G; (iii) AA residue 279 is D; (iv) AA residue 280 is S; (v) AA residue 281 is A, T, or N; (vi) AA residue 307 is S; (vii) AA residue 342 is S or N; (viii) AA residue 345 is A; (ix) AA residue 346 is N or G; (x) AA residue 349 is Q or D; and (xi) AA residue 356 is S or H;

Homologue 19
(SEQ ID NO: 87)
MPTSSRFASQSRVPLPGSERKPFVPAGAPKAAKTPKVSTAVKTVPATGRI
RVSLIVPPKQPLDTKRLGKLDARLSRAQFAARHGADPASVRLVKAFAKEF
GLTVEPITQPGRCTVQLSGT(C/V/D)AAMRKAFAISLVEHTTEQGKFRL
REGEISLPAELEGHVLAVLGLDNRPQAKPHFRIAKPRATNVSYTPVQVAQ
MYGFPAGATATGQTIGIIELGGGYRAADLTAYFKTLGLPAPTVTAVPIDG
GKNTPGNA(N/S/K/G)GADGEVMLDIEVCAAVAQGAKIAVYFTT(N/D)
(T/S)(D/A/T/N)QGFIDAITTAVHDSTNKPSVISISW(G//S)GPESS
WTEQSMTALDAACQAAAAVGVTITVAAGD(N/S)GS(S/T/S)(D/N/G)
GA(S/Q/D)GDNV(D/S/H)FPASSPHVLACGGTKLVGSGSTITSEVVWD
ETSNDEGATGGGVSTVFALPTWQKNANVPSPTTSAGGRGVPDVSGDADPS
TGYTIRVDSETTVIGGTSAVAPLWAGLIALANAQNKVAAGFVNPALYAAG
AKKAFRDITQGNNGSFSAGPGWDACTGLGSPVGNLVIQAVAPKSTTTKKA
KKGKTK,

wherein one, two, three, four, five, six, seven, eight, nine, ten, or all eleven of the following are true: (i) AA residue 121 is V or D; (ii) AA residue 253 is S, K, or G; (iii) AA residue 282 is D; (iv) AA residue 283 is S; (v) AA residue 284 is A, T, or N; (vi) AA residue 310 is S; (vii) AA residue 345 is S; (viii) AA residue 348 is T or A; (ix) AA residue 349 is N or G; (x) AA residue 352 is Q or D; and (xi) AA residue 357 is S or H; and

Homologue 26
(SEQ ID NO: 88)
MHSYLKQQSHMQSYLEQENHMRSYLEMRKKPYFDDLANIRPGGLTPAQVC
QAYQFAKVQPVRPVKLGIVSLAGQYLSSDMSKAFTGYGLPTPVVSTAGSQ
VLGDLWSNVE(N/S/K/G)MMDIEIAGAAWAYATGTAATLLMQFEP(N/
D)(N/T/S)(E/D/A/T/N)TGIPNAINALVAAGCEVISISW(G/S)APA
NLQTMEAITARKEACKQAAVQNVHVFAASGD(E/S/N)SL(N/T/A)(D/
N/G)(G/Q/D)TNSRTP(D/S/H)DPCCDPNVWGVGGTRLVLQADGSIAQ
ESAWGDGNAADKGGGGGFDSREPLPDYQVGVVHSEHRGSPDSSANADPGT
GYAIVANGQWLIGGGTSASAPLTAGYVAAILSTLPGPISQSVLQRKLYTA
HKTAFRDILLGSNGAPARPGWEEATGLGSINGPGLAAALQS.

wherein one, two, three, four, five, six, seven, eight, nine, or all ten of the following are true: (i) AA residue 111 is S, K, or G; (ii) AA residue 139 is D; (iii) AA residue 140 is T or S; (iv) AA residue 141 is D, A, T, or N; (v) AA residue 164 is S; (vi) AA residue 199 is S or N; (vii) AA residue 202 is T or A; (viii) AA residue 203 is N or G; (ix) AA residue 204 is Q or D; and (x) AA residue 211 is S or H.

The polypeptides may be processed versions of the recited polypeptides; the presently claimed polypeptides include any such processed versions of the recited polypeptides. Processed versions of the polypeptides are as defined above.

In one embodiment, the isolated polypeptide comprises the amino acid sequence of a polypeptide selected from Homologues 4, Homolog 6 mutant, and Homologs 13 and 26, or processed versions thereof. In another embodiment, the isolated polypeptide comprises the amino acid sequence of Hom 4 mutant:

(SEQ ID NO: 89)
MANHPLNGSERECLKDAQPIGKADPNERLEVTMLVRRRSHDAFEKHISAL
AAQGASAKHIDHDEFTKHFGADSADLAAVHAFAQKHGLSVVESHEARRAV
VLSGTVAQFDAAFGVSLQQYEHDGGTYRGRTGPIHLPDELNGVVDAVMGL
DNRPQARPSFRTRAQGNVRWTARAAGASTFTPVQLASLYDFPQGDGQNQC
IGIIELGGGYRPADLKTYFASLNMKAPSVTAVSVDHGRNHPTGDPNGPDG
EVMLDIEVAGAVAPGAKIVVYFAPNTDAGFIDAIGTAIHDTKNKPSVISI
SWSGPESAWTQQAMNAFDQAFQSAAALGVTICAASGDNGSGGGVGDGADH
VHFPASSPYALGCGGTSLQASGNGIASETVWNDGANGGATGGGVSSFFAL
PAWQEGLRVTRAGGAHSPLAMRGVPDVAGNADPVTGYEVRVDGHDMVIGG
TSAVAPLWAGLIARINAIKGAPVGYINPHLYKDPLALVDITKGNNDDFHA
TAGWDACTGLGRPDGKKVKDAVS,

or a processed version thereof.

The polypeptides disclosed herein have been identified as having similar, improved, or complementary activity compared to Kumamolisin-related poypeptides in hydrolyzing proline (P)- and glutamine (Q)-rich components of gluten known as ‘gliadins’ believed responsible for the bulk of the immune response in most celiac sprue patients. Numerous other Kumamolisin homologues tested by the inventors possessed little or no such gliadin hydrolyzing activity. Thus, the polypeptides can be used to treat celiac sprue. The polypeptides of this aspect of the invention degrade gliadins at various pHs. Such degradation occurs under the conditions disclosed in the examples that follow.

As used throughout the present application, the term “polypeptide” is used in its broadest sense to refer to a sequence of subunit amino acids, whether naturally occurring or of synthetic origin. The polypeptides of the invention may comprise L-amino acids, D-amino acids (which are resistant to L-amino acid-specific proteases in vivo), or a combination of D- and L-amino acids. The polypeptides described herein may be chemically synthesized or recombinantly expressed. The polypeptides may be linked to other compounds to promote an increased half-life in vivo, such as by PEGylation, HESylation, PASylation, or glycosylation. Such linkage can be covalent or non-covalent as is understood by those of skill in the art. The polypeptides may be linked to any other suitable linkers, including but not limited to any linkers that can be used for purification or detection (such as FLAG or His tags).

In a further aspect, the invention provides compositions, comprising
(a) one or more polypeptides comprising the amino acid sequence of a polypeptide selected from the group consisting of SEQ ID NOs: 74-78, 80-88, 95, 97-99, and 102-111, or processed versions thereof; and
(b) one or more further polypeptides comprising an amino acid sequence selected from the group consisting of:
   (A) an amino acid sequence at least 75%, 80%, 85%, 90%, 95%, or 100% identical to the amino acid sequence of SEQ ID NO:35, wherein
   (i) the polypeptide degrades a PQPQLP (SEQ ID NO:34) peptide at pH 4; and
   (ii) residue 278 is Ser, residue 78 is Glu, and residue 82 is Asp
   (B) an an amino acid sequence at least 75%, 80%, 85%, 90%, 95%, or 100% identical to the amino acid sequence of SEQ ID NO:1, wherein
   (i) the polypeptide degrades a PQPQLP (SEQ ID NO:34) peptide at pH 4; and
   (ii) residue 467 is Ser, residue 267 is Glu, and residue 271 is Asp.

The one or more further polypeptides can be any as described above. For example, the one or more further polypeptides may comprise or consist of an amino acid sequence at least 75%, 80%, 85%, 90%, 95%, or 100% identical to any one of SEQ ID NOS:2-33 or 36-67, or, alternatively, 2-32 or 36-66. In another embodiment, the one or more further polypeptides comprise or consist of KumaMax™ (SEQ ID NO: 90), or a processed version thereof.

In one embodiment, the composition comprises Homologue 4 (SEQ ID NO: 75 or 98) or full length mutant Homologue 4 (SEQ ID NO: 89), or processed versions thereof, together with the one or more of the further polypeptides disclosed herein, including but not limited to KumaMax™ (SEQ ID NO: 90), or a processed version thereof. In another embodiment, the composition comprises SEQ ID NO: 74, SEQ ID NO: 77, and/or SEQ ID NO: 78 (Homologue 1, Homologue 6, and/or the Homologue 6 mutant), or processed versions thereof, together with the one or more of the further polypeptides disclosed herein, including but not limited to KumaMax™ (SEQ ID NO: 90), or a processed version thereof. In a further embodiment, the method comprises administering SEQ ID NO: 88 and/or 111 (Homologue 26) or a processed version thereof, together with the one or more further polypeptides, including but not limited to KumaMax™ (SEQ ID NO: 90), or a processed version thereof.

In another aspect, the present invention provides isolated nucleic acids encoding the polypeptide of any aspect or embodiment of the invention. The isolated nucleic acid sequence may comprise RNA or DNA. As used herein, “isolated nucleic acids” are those that have been removed from their normal surrounding nucleic acid sequences in the genome or in cDNA sequences. Such isolated nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the invention.

In a further aspect, the present invention provides nucleic acid expression vectors comprising the isolated nucleic acid of any embodiment of the invention operatively linked to a suitable control sequence. “Recombinant expression vector” includes vectors that operatively link a nucleic acid coding region or gene to any control sequences capable of effecting expression of the gene product. “Control sequences” operably linked to the nucleic acid sequences of the invention are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type known in the art, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The construction of expression vectors for use in transfecting prokaryotic cells is also well known in the art, and thus can be accomplished via standard techniques. (See, for example, Sambrook, Fritsch, and Maniatis, in. Molecular Cloning, A Laboratory Manual, Cold Spring Harbor Laboratory Press, 1989; Gene Transfer and Expression Protocols, pp. 109-128, ed. E. J. Murray, The Humana Press Inc., Clifton, N.J.), and the Ambion 1998 Catalog (Ambion, Austin, TX). The expression vector must be replicable in the host organisms either as an episome or by integration into host chromosomal DNA. In a preferred embodiment, the expression vector comprises a plasmid. However, the invention is intended to include other expression vectors that serve equivalent functions, such as viral vectors.

In another aspect, the present invention provides recombinant host cells comprising the nucleic acid expression vectors of the invention. The host cells can be either prokaryotic or eukaryotic. The cells can be transiently or stably transfected or transduced. Such transfection and transduction of expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press; Culture of Animal Cells: A Manual of Basic Technique, 2^{nd} Ed. (R. I. Freshney. 1987. Liss, Inc. New York, NY). A method of producing a polypeptide according to the invention is an additional part of the invention. The method comprises the steps of (a) culturing a host according to this aspect of the invention under conditions conducive to the expression of the polypeptide, and (b) optionally, recovering the expressed polypeptide. The expressed polypeptide can be recovered from the cell free extract, cell pellet, or recovered from the culture medium. Methods to purify recombinantly expressed polypeptides are well known to the man skilled in the art.

In a still further aspect, the present invention provides pharmaceutical compositions, comprising the polypeptide, nucleic acid, nucleic acid expression vector, the recombinant host cell, or composition of any aspect or embodiment of the invention, together with a pharmaceutically acceptable carrier. The pharmaceutical compositions of the invention can be used, for example, in the methods of the invention described below. The pharmaceutical composition may comprise in addition to the polypeptides, nucleic acids, etc. of the invention (a) a lyoprotectant; (b) a surfactant; (c) a bulking agent; (d) a tonicity adjusting agent; (e) a stabilizer; (f) a preservative and/or (g) a buffer.

In some embodiments, the buffer in the pharmaceutical composition is a Tris buffer, a histidine buffer, a phosphate buffer, a citrate buffer or an acetate buffer. The pharmaceutical composition may also include a lyoprotectant, e.g. sucrose, sorbitol or trehalose. In certain embodiments, the pharmaceutical composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the pharmaceutical composition includes a bulking agent, like glycine. In yet other embodiments, the pharmaceutical composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

The polypeptides, nucleic acids, etc. of the invention may be the sole active agent in the pharmaceutical composition, or the composition may further comprise one or more other active agents suitable for an intended use.

The pharmaceutical compositions described herein generally comprise a combination of a compound described herein and a pharmaceutically acceptable carrier, diluent, or excipient. Such compositions are substantially free of non-pharmaceutically acceptable components, i.e., contain amounts of non-pharmaceutically acceptable components lower than permitted by US regulatory requirements at the time of filing this application. In some embodiments of this aspect, if the compound is dissolved or suspended in water, the composition further optionally comprises an additional pharmaceutically acceptable carrier, diluent, or excipient. In other embodiments, the pharmaceutical compositions described herein are solid pharmaceutical compositions (e.g., tablet, capsules, etc.).

These compositions can be prepared in a manner well known in the pharmaceutical art, and can be administered by any suitable route. In a preferred embodiment, the pharmaceutical compositions and formulations are designed for oral administration. Conventional pharmaceutical carriers, aqueous, powder or oily bases, thickeners and tine like may be necessary or desirable.

The pharmaceutical compositions can be in any suitable form, including but not limited to tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

### EXAMPLES

Celiac disease is an autoimmune disorder that afflicts approximately 1% of the population (1, 2). This disease is characterized by an inflammatory reaction to gluten, the major protein in wheat flour, and to related proteins in barley and rye (2). Gluten is composed of a heterogeneous mixture of the glycoproteins gliadin and glutenin (3). Upon ingestion, a-gliadin is partially degraded by gastric and intestinal proteases to oligopeptides, which are resistant to further proteolysis due to their unusually high proline and glutamine content (3). Immunogenic oligopeptides that result from incomplete proteolysis are enriched in the PQ motif (4, 5), which stimulate inflammation and injury in the intestine of people with Celiac disease. Currently the only treatment for this disease is complete elimination of gluten from the diet, which is difficult to attain due to the ubiquity of this protein in modern food products (6).

Oral enzyme therapy (OET) in which orally administered proteases are employed to hydrolyze immunogenic peptides before they are capable of triggering inflammation is currently being explored as a treatment for gluten intolerance. For this purpose, several different proteases have been considered due to their specificity for cleavage after either proline or glutamine residues. However, these enzymes often demonstrate characteristics that hinder their use in OET for gluten degradation. Most of these peptidases exhibit optimal catalytic activity at neutral pH; however, the pH of the human stomach ranges from 2 to 4. These enzymes are therefore most active when they reach the pH-neutral small intestine, which is too late for effective prevention of Celiac disease as this is the site where gluten-derived pathology develops. Additionally, several of these enzymes demonstrate instability in the low pH of the human stomach, are susceptible to proteolysis by digestive proteases, or require extensive refolding procedures during their purification, which are all characteristics that hamper efforts for clinical use.

The ideal protease for the application of OET in the treatment of gluten intolerance would combine the following traits: optimal activity at low pH, easy purification, stability under the conditions of the human stomach, and high specificity for amino acid motifs found in gluten-derived immunogenic oligopeptides. We previously identified a protease that is highly active in acidic conditions, Kumamolisin-As (KumaWT) from the acidophilic bacterium Alicyclobacillus sendaiensis, and used computational modeling tools to engineer it toward the desired oligopeptide specificity. An exemplary computationally designed enzyme, designated KumaMax™, exhibited over 100-fold increased proteolytic activity and an 800-fold switch in substrate specificity for the targeted PQ motif compared to wild-type KumaWT. In addition, KumaMax™ demonstrates resistance to common gastric proteases and is produced at high yields in E. coli without the need for refolding. The previously designed proteins were assessed for catalytic activity against a PQLP (SEQ ID NO: 68) peptide; exemplary results are provided in Table 2

TABLE 2

| | Fold Change in |
|---|---|
| | Activity of |
| | PQ Hydrolysis |
| | Relative to |
| Mutations to Wild Type Kumamolysin-As | Wild Type |
| (Preprocessed) | Kumamolysin-As |
| Wild Type (WT) | 1.0 |
| T357A | 2.0 |
| G319S, D368S | 2.0 |
| D358G | 3.0 |
| D293A | 3.0 |
| D358N | 4.0 |
| G319S, S354N, D358G, D368H | 5.0 |
| D358G, D368H | 6.0 |
| G319S, D358G, D368H | 7.0 |
| N291D, Q361D | 7.5 |
| S354N, D358G, D368H | 9.0 |
| N291D | 10.0 |
| N291D, D293A, Q361D, D358N | 14.8 |
| N291D, D293A | 15.0 |
| N291D, D293A, D358G, Q.361D | 15.0 |
| N291D, D358N | 18.9 |
| N291D, Q361D, D358G | 20.0 |
| N291D, G319S, D358G, Q361D, D368H | 23.1 |
| N291D, D293A, D358N | 24.0 |
| S262G, T292S, N291D, G319S, D358G, D368H | 29.0 |
| N291D, D293A, G319S, D358G, Q361D, D368H | 40.9 |
| T292S, N291D, G319S, D358G, D368H | 49.0 |
| N291D, G319S, S354N, D358G, Q361D, D368H | 50.0 |
| N291D, G319S, S354N, D358G, D368H | 54.6 |
| N291D, D293A, G319S, S354N, D358G, Q361D, | 58.0 |
| D368H | |
| D293T, N291D, G319S, D358G, D368H | 58.0 |
| S262K, D293N, N291D, G319S, D358G, D368H | 62.0 |
| N291D, G319S, D358G, D368H | 93.0 |
| V119D, S262K, D293T, N291D, G319S, D358G, | 120.0 |
| D368H | |

In the present study, the inventors tested a large number of Kumamolisin homologues obtained from a wide variety of organisms for activity in degrading gliadin proteins. They share the catalytic triad present in Kumamolisin-As (Ser⁴⁶¹-Glu²⁶⁷-Asp²⁷¹ in the Kumamolisin-As pre-processed form). In order to assess the relative abilities of these homologues to target gluten, the purified homologue were incubated with protein with purified peptides that represent the immunogenic regions throughout gliadin, which is the problematic fraction of gluten for celiac patients.

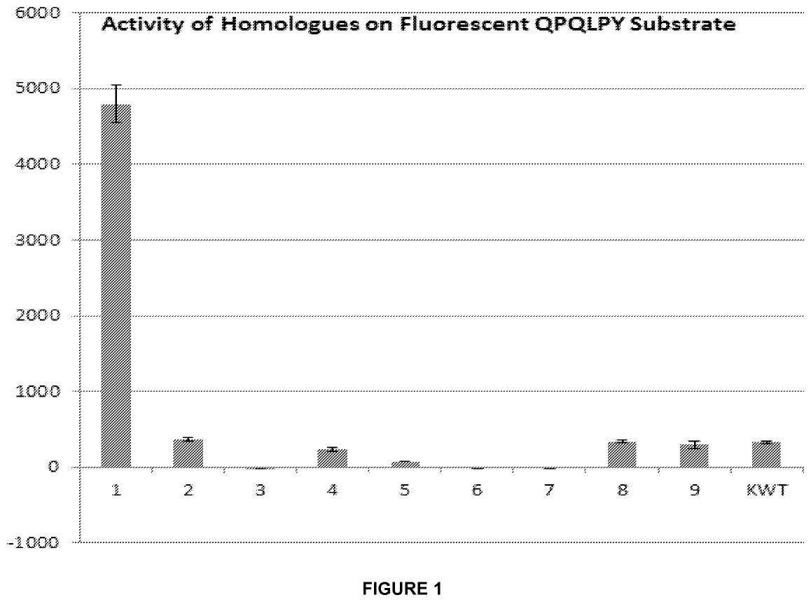

Homologues were assessed for their ability to break down a fluorescent analogue of gliadin, a hexapeptide (QPQLPY (SEQ ID NO: 91)) that was conjugated to a fluorophore and a quencher, in simulated lab gastric conditions (NaOAc buffer pH 4.0 at 37° C.). The rate of degradation can be calculated from measurement of the fluorescence signal over time. The activity was compared to that of Kumamolisin (denoted below as KWT). Kumamolisin has some activity breaking down these gliadin substrates. Exemplary results are shown in FIG. 1. As can be noted, only a subset of the homologues (1, 2, 4, 8, and 9) tested had activity comparable to or better than Kumamolisin under these conditions.

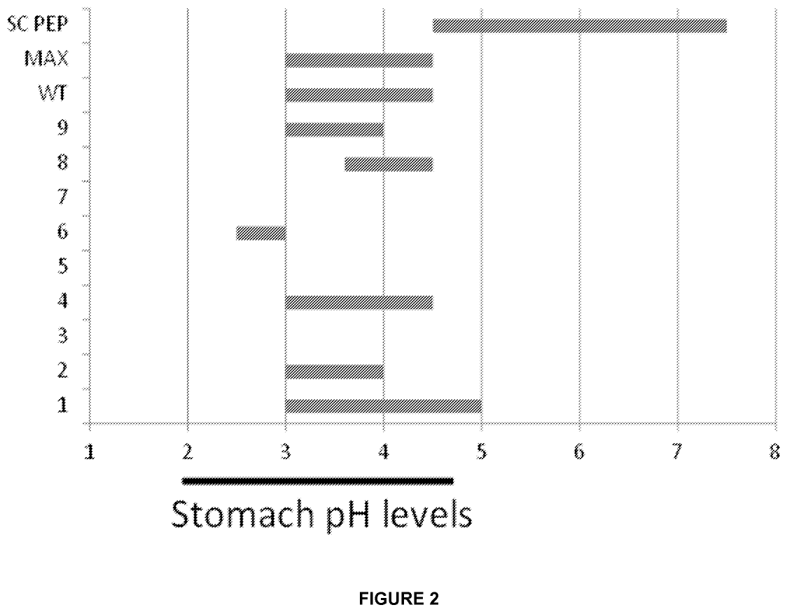

In a further study, the pH levels were varied and the homologues tested for activity at the different pH levels. The data is shown in FIG. 2. Most of the homologues tested demonstrated activities within the pH range of both Kumamolisin (designated below as “WT”) and KumaMax™ (designated as “Max”). Interestingly, two of the homologues had expanded pH ranges compared to Kumamolisin. Homologue 1, was optimally active through pH 5, and Homologue 6 demonstrated optimal activity at a pH level below that of the other homologues and Kumamolisin/KumaMax, explaining why no activity was seen for this homologue in the fluorescent experiment conducted at pH 4. This indicates that homologues 1 and 6 could be used, for example, to expand the pH profile of Kumamolisin-related polypeptides, to more closely mimic pH conditions in the stomach.

We further tested the ability of these homologues to break down different non-fluorescently-labeled peptides that had been linked to celiac disease. Results are provided in Table 3; + and − represents a visual indication of the homologue's ability to break down the indicated peptide: −<+/−<++/−<+<++<+++<++++. The number is the % of peptide that is degraded by the homologue after an 80-mm incubation (so the smaller the number, the more effective the homologue); (undetect) means that the peptide was below detection limit after 80 mm.

As can be seen, Kumamolisin and KumaMax™ have fairly low levels of activity against the γ-gliadin peptide, while Homologue 4 has increased activity against this peptide, suggesting that Homologue 4, alone or in combination therapy with Kumamolisin-related polypeptides, may be an effective therapy for gluten digestion.

TABLE 3

| | γ-gliadin | α2-gliadin | α9-gliadin | Glia_56-79 |
|---|---|---|---|---|
| | (IQPQQPA | (PQPQLPYSQPQ | (QLQPFPQPQL | (LQLQPFPQPQLPYPQP |
| Homologue | QL)(SEQ | PFR)(SEQ ID | PY)(SEQ ID | QLPY)(SEQ ID NO: 94) |
| # | ID NO: 92) | NO: 93) | NO: 70) | |
| 1 | ++/−; 29% | ++(undetect) | −; 80% | ++; 1.6% |
| 2 | +/−; 65% | ++(undetect) | ++/−; 37%; | ++; 0.2% |
| 3 | −; 108% | −; 123% | −; 116% | −; 104% |
| 4 | +; 12% | ++(undetect) | ++/−; 25%; | ++; 0.6% |
| 5 | ++/−; 40% | ++(undetect) | ++/−; 34%; | ++; 0.4% |
| 6 | −; 102% | −; 87% | −; 114% | −; 85% |
| 7 | −; 106% | −; 109% | −; 115% | −; 101% |
| 8 | −; 92% | ++(undetect) | −; 105% | ++/−; 34% |
| 9 | ++/−; 32% | ++(undetect) | +/−; 62% | +; 9% |
| Kumamolisin | ++/−; 39% | ++(undetect) | ++; 2%; | +++; 0.2% |
| (WT) | | | | |
| Max | +/−; 55% | +/−; 65%; | ++; 0.9% | ++++ |

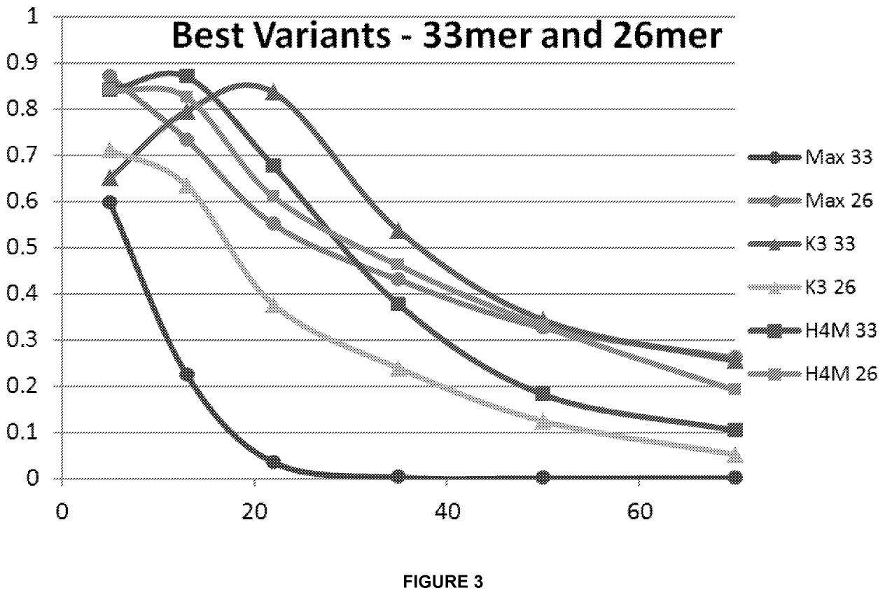

Since these are homologues of Kumamolisin with a low percentage of sequence identity, we made the same mutations in homologues as were made to Kumamolisin in order to generate KumaMax. We tested the activities of these homologues on two peptides that are degradation products of gluten in the stomach and have been specifically linked to celiac disease: the 33mer peptide (LQLQPFPQPQLPYPQPQLPYPQPQLPYPQPQPF (SEQ ID NO: 72)) and the 26mer peptide (FLQPQQPFPQQPQQPYPQQPQQPFPQ (SEQ ID NO: 73)). In particular, the 33mer peptide has been strongly linked to celiac disease. Additionally, the KumaMax™ mutations in the homologues inspired us to make these three active-site mutations alone on the Kumamolisin background (note that KumaMax™ contains a total of 7 mutations from Kumamolisin, but only 3 are within the active site). This mutant, which only contains these active-site mutations, is called the K3 mutant below. The data are provided in Table 4 and FIG. 3. We found that several these homologues demonstrated activity against these two very important peptides, and interestingly, that on Homologue 4 (SEQ ID NO: 75) the KumaMax™ (SEQ ID NO: 90) mutations increased activity against both peptides; in fact, the Homologue 4 Mutant (SEQ ID NO: 89) is the best overall enzyme tested in the experiment shown below, which includes KumaMax™ (SEQ ID NO: 90). This also shows that the K3 mutant could also be combined with KumaMax™ (SEQ ID NO: 90) to generate a more potent therapeutic. (NA=No Activity).

TABLE 4

| Homologue | 33mer | 26mer |
|---|---|---|
| KumaMax | 0.1% | 26% |
| K3 Mutant | 26% | 5% |
| KumaWT | 18% | NA |
| Hom #1 | 46% | NA |
| Hom # 1 Max | NA | NA |
| Hom #2 | 31% | NA |
| Hom #2 Max | 29% | NA |
| Hom #4 | 38% | NA |
| Hom #4 Max | 10% | 19% |
| Hom #5 | NA | NA |
| Hom #5 Max | NA | NA |
| Hom #9 | NA | 52% |
| Hom #9 Max | NA | 60% |
| Hom #10 | NA | NA |
| Hom #10 Max | NA | NA |

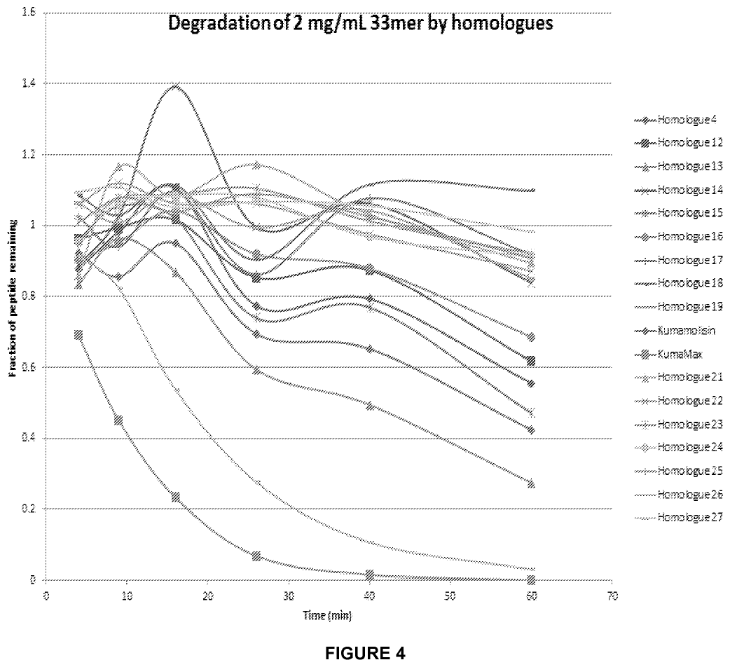

Further studies were done using additional homologues of Kumamolisin. A subset of these homologues (homologues 13 and 26) demonstrated significant activity against the 33mer peptide; see FIG. 4. In particular, homologue 26 in its wild type form had very strong activity comparable to KumaMax™ at breaking down the 33mer peptide.

We then looked at the profile of digested 33mers via HPLC after a 60 minute incubation: degrading the 33-mer at more than one location provides a significant therapeutic advantage. Wild type Kumamolysin degrades the 33-mer down at only a single location (WT-like), while KumaMax™ degrades the 33-mer at multiple locations (Max-like). The data are shown in Table 5.

TABLE 5

| Homologue | Degradation pattern of 33mer (60 min) |
|---|---|
| H4 | WT-like |
| H12 | WT-like |
| H13 | Max-like |
| H14 | WT-like - minor |
| H15 | WT-like |
| H16 | WT-like |
| H17 | WT-like - minor |
| H18 | Undegraded |
| H19 | WT-like - minor |
| H21 | Undegraded |
| H22 | Undegraded |
| H23 | Undegraded |
| H24 | Undegraded |
| H25 | Undegraded |
| H26 | Max-like |
| H27 | Undegraded |

An overall summary of the data is that several homologues, including Homologue 4 (SEQ ID NO:75) and the Homologue 4 mutant (SEQ ID NO: 89)), 13, and 26, can be used as therapeutics to treat celiac disease. Homologue 26 (SEQ ID NOL 88) is almost as potent as KumaMax™ (SEQ ID NO: 90) and this is in the absence of any engineering, and Homologue 4 (demonstrated increased activity with the mutations that were made to KumaMax™. Furthermore, pH profiles from these homologues (in particular, Homologue 6 (including the Homologue 6 mutant) and Homologue 1) suggest that these homologues, alone or in combination, can expand the pH range of therapeutic efficacy in the human stomach.

### METHODS

### Protein Expression and Purification

The genes encoding each protein of interest, harbored in the pET29b plasmid, were transformed into Escherichia coli BL21 (DE3) cells. Individual colonies were picked, inoculated into Terrific Broth™ with 50 μg/μL Kanamycin (TB+Kan), and incubated overnight at 37° C. 500 uL of the overnight culture was added to 500 mL autoinduction media (5 g tryptone, 2.5 g yeast extract, 465 mL ddH₂O), and shaken at 37° C. for roughly 4 hours, then the autoinduction components were added (500 uL MgSO₄, 500 uL 1000× trace metals, 25 mL 20× NPS, 10 mL 20×5052, 500 uL 50 mg/mL Kan). The cultures were then shaken at 18° C. for 30 hours before being spun down. Pellets were resuspended in 10 mL 1× PBS, then lysed via sonication with 5 mL lysis buffer (50 mM HEPES, 500 mM NaCl, 1 mM bME, 2 mg/mL lysozyme, 0.2 mg/mL DNase, ddH₂O) and spun down. The proteins were then purified over 1 mL TALON cobalt affinity columns. KumaMax, KumaWT, and SC Pep were washed three times with 20 mL wash buffer (10 mM imidazole, 50 mM HEPES, 500 mM NaCl, 1 mM bME, ddH₂O), and then eluted in 15 mL of elution buffer (200 mM imidazole, 50 mM HEPES, 500 mM NaCl, 1 mM bME). EP-B2 had to be refolded on the column, so after lysis the pellets were resuspended in 10 mL of EP-B2 buffer, which differs from the wash buffer only in that it is diluted in guanidine hydrochloride instead of ddH₂O to allow for denaturation of the EP-B2 inclusion bodies. This resuspension was pelleted, and the supernatant (containing denatured EP-B2) was filtered with a 0.8 μm filter onto the column. EP-B2 was washed once with 20 mL of the EP-B2 buffer, before being washed twice with 20 mL of the wash buffer to refold the protein on the column. Protein was eluted with 15 ml of the elution buffer. All proteins were concentrated from 15 mL down to ˜500 uL, then dialyzed once in 1 L dialysis buffer (20% glycerol, 50 mM HEPES, 500 mM NaCl, 1 mM bME). Protein concentration was calculated spectrophotometrically with extinction coefficients of 53,985 M⁻¹cm⁻¹ for KumaWT and all KumaWT variants, 152,290 M⁻¹cm⁻¹ for SC Pep, and 58,245 M⁻¹cm⁻¹ for EP-B2.

### Purified Enzyme Assay

The variants of Kumamolisin-As that displayed the most activity on the FQ substrate in the activity screen were sequenced, then purified in small scale. 500 uL of TB+Kan overnight cultures were added to 50 mL TB+Kan and grown at 37 °C. until reaching an optical density of 0.5-0.8. IPTG was added to 0.5 mM, and the cultures were expressed at 22° C. for 16-24 hours. The cells were spun down, resuspended in 500 uL of wash buffer (1× PBS, 5 mM imidazole, ddH₂O), transferred to a 2 mL Eppendorf tube, and lysed in 1 mL lysis buffer (1× PBS, 5 mM imidazole, 2× Bug Buster™, 2 mg/mL lysozyme, 0.2 mg/mL DNase, ddH₂O). After centrifugation, the supernatant was decanted into a fresh tube. Columns with 200 uL of TALON cobalt resin were placed in Eppendorf tubes, and the supernatant was poured over the columns and rocked for 20 minutes before spinning down and discarding the flow-through. The proteins were washed three times with 500 uL wash buffer, discarding the flow-through between washes. Enzymes were eluted in 200 uL elution buffer (1× PBS, 200 mM imidazole, dd H₂O), and concentrations were calculated spectrophotometrically using an extinction coefficient of 53,985 M⁻¹cm⁻¹ .

For the assay, the Kumamolisin-As mutants were incubated for 15 minutes in pH 4 100 mM sodium acetate buffer. Enzyme was added to 5 μM substrate so that the final protein concentration was 0.0125 mg/mL. The fluorescence was measured at 30-second intervals for 1 hour.

### Kinetic Characterization

Enzyme variant proclivity for gluten degradation was measured by hydrolysis of the fluorescently quenched α-gliadin hexapeptide analogue QXL520-PQPQLP-K(5-FAM)-NH2 (FQ) (SEQ ID NO: 69) as a substrate. Each enzyme was incubated at room temperature for 15 minutes in 100 mM pH 4 sodium acetate buffer. After 15 minutes, 50 uL of fluorescent substrate was added ranging in final concentration between 100, 50, 25, 12.5, 6.25, and 0 μM peptide, and maintaining concentrations of 0.05 μM KumaMax™, 0.5 μM KumaWT, 0.5 μM SC Pep, and 0.5 μM EP-B2 across all variations in substrate concentration. The plate was read immediately on the spectrophotometer for an hour, using 455 nm wavelength for excitation and reading 485 nm wavelength for emission.

The enzymes were also tested for specificity to different dipeptide motifs using a variety of chromogenic substrates that release p-nitroaniline (pNA) upon hydrolysis: [Suc-APQ-pNA], [Suc-AQP-pNA], [Suc-APE-pNA], and [Suc-APR-pNA]. Again, each enzyme was incubated at room temperature for 15 minutes in 100 mM pH 4 sodium acetate buffer. After 15 minutes, 20 uL of substrate was added to the enzyme incubation so that the final concentrations of substrate ranged between 1000, 500, 250, 125, 62.5, 31.25, 15.625, and 0 μM, and all enzymes being tested ended in a concentration of 0.5 μM. The plate was read immediately on the spectrophotometer for an hour, monitoring absorption by the reactions at 385 nm.

The standard curve for the fluorescent peptide involved mixing substrate and product together at varying concentrations in pH 4 buffer. Substrate concentrations were 100, 50, 25, 12.5, 6.25, and 0 μM, and product concentrations were 20, 5, 1.25, 0.3125, 0.078125, 0 μM.

The standard curve for the absorbent peptide involved product concentrations of 100, 50, 25, 12.5, 6.25, 3.125, 1.5625, 0.78125, 0.390625, 0.1953125, 0.09765625, and 0 μM diluted in pH 4 buffer.

### Protease Stability

Enzyme stability was determined in the presence the digestive proteases, pepsin and trypsin. KumaWT, KumaMax™, SC Pep, and EP-B2 were incubated in buffer matching the native pH environment of each digestive protease. pH 3.5 100 mM sodium acetate was used to pre-incubate the enzymes for pepsin digestion assays, and pH 7.5 dialysis buffer (see “Protein Expression and Purification”) for the trypsin digestion assays. Each experimental enzyme was incubated at 37° C. for 15 minutes in each buffer, at a concentration of 0.2 mg/mL.

After pre-incubation in the appropriate buffer, 0.1 mg/mL digestive protease was added. The reactions were done in triplicate, and were incubated at 37° C. for 30 minutes. Adding SDS and boiling for 5 minutes ensured digestive protease inactivation. An SDS-PAGE gel allowed quantification of enzyme degradation, using ImageJ.

The rate of protein self-proteolysis was determined at pH 4 and 7.5 in the absence of pepsin or trypsin. Each enzyme, at a concentration of 0.2 mg/mL, was incubated in pH 4 100 mM sodium acetate and pH 7.5 dialysis buffer. At 20, 40, and 60 minutes, timepoints were taken. SDS was added, and the aliquots were boiled for 5 minutes to ensure denaturation of the enzymes and inhibition of further self-proteolysis. Again, an SDS-PAGE gel in conjunction with ImageJ allowed quantification of enzyme self-proteolysis.

### LCMS Gliadin Degradation Assay

Enzyme activity on full-length α9-gliadin was measured using high-performance liquid-chromatography mass spectrometry. For each enzyme, 7 μL of pH 4 1M sodium acetate buffer was added to 28 μL of 5 μM enzyme, and incubated alongside separate tubes of 3 μL gliadin at 37° C. for 15 minutes. Next 27 μL of each enzyme mixture, and 27 μL of dialysis buffer as a control, were added to each tube of gliadin. These were incubated once more at 37° C., and 5 μL samples were taken at 10, 20, 30, 40, and 50 minutes. Each timepoint sample was quenched in 95 μL of 80% acetonitrile with 1% formic acid and approximately 33 μM leupeptin. The samples were analyzed on the HPLC to compare gliadin degradation by the different proteases over time.

### REFERENCES

1. Armstrong, M. J., Hegade, V. S. & Robins, G. Advances in coeliac disease. Curr Opin Gastroenterol 28, 104-12 (2012).
2. Sollid, L. M. Coeliac disease: dissecting a complex inflammatory disorder. Nat Rev Immunol 2, 647-55 (2002).
3. Wieser, H. Chemistry of gluten proteins. Food Microbial 24, 115-9 (2007).
4. Shan, L. et al. Structural basis for gluten intolerance in celiac sprue. Science 297, 2275-9 (2002).
5. Shan, L. Identification and analysis of multivalent proteolyticaily resistant peptides from gluten: implications for celiac sprue. Journal of Proteome Research (2005).
6. Chand, N. & Mihas, A. A. Celiac disease: current concepts in diagnosis and treatment. J Clin Gastroenterol 40, 3-14 (2006).
7. Shan, L., Marti. T., Sollid, L. M., Gray, G. M. & Khosla, C. Comparative biochemical analysis of three bacterial prolyl endopeptidases: implications for coeliac sprue. Biochem J 383, 311-8 (2004).
8. Siegel, M. et al. Rational design of combination enzyme therapy for celiac sprue. Chem Biol 13, 649-58 (2006).
9. Stepniak, D. et al. Highly efficient gluten degradation with a newly identified prolyl endoprotease: implications for celiac disease. Am J Physiol Gastrointest Liver Physiol 291, G621-9 (2006).
10. Ehren, J. et al. A food-grade enzyme preparation with modest gluten detoxification properties. PLoS One 4, e6313 (2009).
11. Bethune, M. T., Strop, P., Tang, Y., Sollid, L. M. & Khosla, C. Heterologous expression, purification, refolding, and structural-functional characterization of EP-B2, a self-activating barley cysteine endoprotease. Chem Biol 13, 637-47 (2006).
12. Okubo, A. et al, Processing, catalytic activity and crystal structures of kumamolisin-As with an engineered active site. FEBS J 273, 2563-76 (2006).
13. Gardner, J. D., Ciociola, A. A, & Robinson, M. Measurement of meal-stimulated gastric acid secretion by in vivo gastric autotitration. J Appl Physiol 92, 427-34 (2002).
14. Wlodawer, A. et al. Crystallographic and biochemical investigations of kumamolisin-As, a serine-carboxyl peptidase with collagenase activity. J Biol Chem 279, 21500-10 (2004).
15. Ehren, J., Govindarajan, S., Moron, B., Minshull, J. & Khosla, C. Protein engineering of improved prolyl endopeptidases for celiac sprue therapy. Protein Eng Des Sel 21, 699-707 (2008).
16. Bethune, M. T. & Khosla, C. Oral enzyme therapy for celiac sprue. Methods Enzymol 502, 241-71 (2012).
17. Gass, J., Vora, H., Bethune, M. T., Gray, G. M. & Khosla, C. Effect of barley endoprotease EP-B2 on gluten digestion in the intact rat. J Pharmacol Exp Ther 318, 1178-86 (2006).
18. Vora, H., McIntire, J., Kumar, P., Deshpande, M. & Khosla, C. A scaleable manufacturing process for pro-EP-B2, a cysteine protease from barley indicated for celiac sprue. Biotechnol Bioeng 98, 177-85 (2007).

1.-29. (canceled)

30. An isolated polypeptide selected from the following group, or analogs thereof:
(a) an isolated polypeptide comprising an amino acid sequence, wherein the amino acid sequence comprises one, two, three, four, five, six, seven, eight, nine, ten, or eleven of the following substitutions from SEQ ID NO: 97: (i) V or D at AA residue 116; (ii) S, K, or G at AA residue 255; (iii) D at AA residue 284; (iv) T at AA residue 285; (v) A, T, or N at AA residue 286; (vi) S at AA residue 312; (vii) N at AA residue 347; (viii) T or A at AA residue 350; (ix) N or G at AA residue 351; (x) D at AA residue 354; and (xi) S or H at AA residue 361;
(b) an isolated polypeptide comprising an amino acid sequence, wherein the amino acid sequence comprises one, two, three, four, five, six, seven, eight, nine, ten, or eleven of the following substitutions from SEQ ID NO: 98: (i) D at AA residue 106; (ii) S, K, or G at AA residue 246; (iii) D at AA residue 275; (iv) S at AA residue 276; (v) A, T, or N at AA residue 277; (vi) S at AA residue; (vii) S at AA residue 338; (viii) T or A at AA residue 341; (ix) N or G at AA residue 342; (x) Q or D at AA residue 345; and (xi) S or H at AA residue 352;
(c) an isolated polypeptide comprising an amino acid sequence, wherein the amino acid sequence comprises one, two, three, four, five, six, seven, eight, nine, ten, or eleven of the following substitutions from SEQ ID NO: 99: (i) D at AA residue 120; (ii) S, K, or G at AA residue 259; (iii) D at AA residue 288; (iv) T at AA residue 289; (v) A, T, N at AA residue 290; (vi) A at AA residue 316; (vii) S or N at AA residue 351; (viii) A at AA residue 354; (ix) N or G at AA residue 355; (x) D at AA residue 358; and (xi) S or H at AA residue 365;
(d) an isolated polypeptide comprising an amino acid sequence as set forth in SEQ ID NO: 78;
(e) an isolated polypeptide comprising an amino acid sequence, wherein the amino acid sequence comprises one, two, three, four, five, six, seven, eight, nine, ten, or eleven of the following substitutions from SEQ ID NO: 102: (i) V or D at AA residue 107; (ii) S, K, or G at AA residue 245; (iii) AA residue 274 is D; (iv) AA residue 275 is T; (v) AA residue 276 is A, T, or N; (vi) AA residue 302 is S; (vii) AA residue 337 is S or N; (viii) AA residue 340 is T or A; (ix) AA residue 341 is N or G; (x) AA residue 344 is Q or D; and (xi) AA residue 351 is S or H;
(f) an isolated polypeptide comprising an amino acid sequence, wherein the amino acid sequence comprises one, two, three, four, five, six, seven, eight, nine, ten, or eleven of the following substitutions: (i) V or D at AA residue 76; (ii) S, K, or G at AA residue 206; (iii) D at AA residue 235; (iv) S at AA residue 236; (v) A, T, N at AA residue 237; (vi) S at AA residue 262; (vii) S or N at AA residue 297; (viii) T or A at AA residue 300; (ix) N or G at AA residue 301; (x) Q or D at AA residue 302; and (xi) S or H at AA residue 309;
(g) an isolated polypeptide comprising an amino acid sequence, wherein the amino acid sequence comprises one, two, three, four, five, six, seven, eight, nine, ten, or eleven of the following substitutions from SEQ ID NO: 104: (i) D at AA residue 105; (ii) S or K at AA residue 244; (iii) D at AA residue 272; (iv) S at AA residue 273; (v) A, T, or N at AA residue 274; (vi) S at AA residue 299; (vii) N at AA residue 334; (viii) T or A at AA residue 337; (ix) N or G at AA residue 338; (x) Q or D at AA residue 341; and (xi) S or H at AA residue 348;
(h) an isolated polypeptide comprising an amino acid sequence, wherein the amino acid sequence comprises one, two, three, four, five, six, seven, eight, nine, ten, or eleven substitutions from SEQ ID NO: 105: (i) V or D at AA residue 106; (ii) S, K, or G at AA residue 244; (iii) D at AA residue 273; (iv) T at AA residue 274; (v) A, T, N at AA residue 275; (vi) S at AA residue 301; (vii) N at AA residue 336; (viii) T or A at AA residue 339; (ix) N or G at AA residue 340; (x) D at AA residue 343; and (xi) S or H at AA residue 350;
(i) an isolated polypeptide comprising an amino acid sequence, wherein the amino acid sequence comprises one, two, three, four, five, six, seven, eight, nine, ten, or eleven of the following substitutions from SEQ ID NO: 106: (i) V or D at AA residue 107; (ii) S, K, or G at AA residue 245; (iii) AA residue 274 is D; (iv) AA residue 275 is T; (v) AA residue 276 is A, T, or N; (vi) AA residue 302 is S; (vii) AA residue 337 is N; (viii) AA residue 340 is T or A; (ix) AA residue 341 is N or G; (x) AA residue 344 is Q or D; and (xi) AA residue 351 is S or H;
(j) an isolated polypeptide comprising an amino acid sequence, wherein the amino acid sequence comprises one, two, three, four, five, six, seven, eight, nine, ten, or eleven of the following substitutions from SEQ ID NO: 107: (i) V or D at AA residue 104 is V or D; (ii) S, K, or G at AA residue 241; (iii) D at AA residue 270; (iv) S at AA residue 271; (v) D, A, T, or N at AA residue 272; (vi) S at AA residue 398; (vii) S at AA residue 33; (viii) A at AA residue 336; (ix) N or G at AA residue 337; (x) D at AA residue 340; and (xi) S or H at AA residue 347;
(k) an isolated polypeptide comprising an amino acid sequence, wherein the amino acid sequence comprises one, two, three, four, five, six, seven, eight, nine, ten, or eleven of the following substitutions from SEQ ID NO: 108: (i) D at AA residue 104; (ii) S, K, or G at AA residue 245; (iii) D at AA residue 274; (iv) S at AA residue 275; (v) A, T, or N at AA residue 276; (vi) S at AA residue 302; (vii) S or N at AA residue 337; (viii) T or A at AA residue 340; (ix) N or G at AA residue 341; (x) Q or D at AA residue 344; and (xi) S or H at AA residue 351;
(l) an isolated polypeptide comprising an amino acid sequence, wherein the amino acid sequence comprises one, two, three, four, five, six, seven, eight, nine, ten, or eleven of the following substitutions from SEQ ID NO: 109: (i) V or D at AA residue 118; (ii) K or G at AA residue 250; (iii) D at AA residue 279; (iv) S at AA residue 280; (v) A, T, or N at AA residue 281; (vi) S at AA residue 307; (vii) S or N at AA residue 342; (viii) A at AA residue 345; (ix) N or G at AA residue 346; (x) Q or D at AA residue 349; and (xi) S or H at AA residue 356;
(m) an isolated polypeptide comprising an amino acid sequence, wherein the amino acid sequence comprises one, two, three, four, five, six, seven, eight, nine, ten, or eleven of the following substitutions from SEQ ID NO: 110: (i) V or D at AA residue 121; (ii) S, K, or G at AA residue 253; (iii) D at AA residue 282; (iv) S at AA residue 283; (v) A, T, or N at AA residue 284; (vi) S at AA residue 310; (vii) S at AA residue 345; (viii) T or A at AA residue 348; (ix) N or G at AA residue 349; (x) Q or D at AA residue 352; and (xi) S or H at AA residue 357;
(n) an isolated polypeptide comprising an amino acid sequence, wherein the amino acid sequence comprises one, two, three, four, five, six, seven, eight, nine, or ten of the following substitutions from SEQ ID NO: 111: (i) S, K, or G at AA residue 111; (ii) D at AA residue 139; (iii) T or S at AA residue 140; (iv) D, A, T or N at AA residue 141; (v) S at AA residue 164; (vi) S or N at AA residue 199; (vii) T or A at AA residue 202; (viii) N or G at AA residue 203; (ix) Q or D at AA residue 204; and (x) S or H at AA residue 211; and
(o) an isolated polypeptide comprising an amino acid sequence as set forth in SEQ ID NO:89.

31. The isolated polypeptide of claim 30, selected from the group consisting of the following, or analogs thereof:
(i) an isolated polypeptide comprising an amino acid sequence, wherein the amino acid sequence comprises one, two, three, four, five, six, seven, eight, nine, ten, or eleven of the following substitutions from SEQ ID NO: 98: (i) D at AA residue 106; (ii) S, K, or G at AA residue 246; (iii) D at AA residue 275; (iv) S at AA residue 276; (v) A, T, or N at AA residue 277; (vi) S at AA residue 303; (vii) S at AA residue 338; (viii) Tor A at AA residue 341; (ix) N or G at AA residue 342; (x) Q or D at AA residue 345; and (xi) S or H at AA residue 352;
(ii) an isolated polypeptide comprising an amino acid sequence as set forth in SEQ ID NO: 78;
(iii) an isolated polypeptide comprising an amino acid sequence, wherein the amino acid sequence comprises one, two, three, four, five, six, seven, eight, nine, ten, or eleven substitutions from SEQ ID NO: 105: (i) V or D at AA residue 106; (ii) S, K, or G at AA residue 244; (iii) D at AA residue 273; (iv) T at AA residue 274; (v) A, T, or N at AA residue 275; (vi) S at AA residue 301; (vii) N at AA residue 336; (viii) T or A at AA residue 339; (ix) N or G at AA residue 340 is; (x) D at AA residue 343; and (xi) S or H at AA residue 350;
(iv) an isolated polypeptide comprising an amino acid sequence, wherein the amino acid sequence comprises one, two, three, four, five, six, seven, eight, nine, or ten substitutions from SEQ ID NO: 111: (i) S, K, or G at AA residue 111; (ii) D at AA residue; (iii) T or S at AA residue 140; (iv) AA D, A, T, or N at residue 141; (v) S at AA residue 164; (vi) S or N at AA residue 199; (vii) T or A at AA residue 202; (viii) N or G at AA residue 203; (ix) Q or D at AA residue 204; and (x) S or H at AA residue 211; and
(v) an isolated polypeptide comprising an amino acid sequence as set forth in SEQ ID NO:89.

32. The isolated polypeptide of claim 31, comprising the amino acid sequence of SEQ ID NO: 78, the amino acid sequence of SEQ ID NO: 89, or an analog thereof.

33. A nucleic acid encoding the polypeptide of claim 30.

34. A nucleic acid expression vector comprising the nucleic acid of claim 33.

35. A recombinant host cell comprising the nucleic acid expression vector of claim 34.

36. A composition, comprising
(a) the isolated polypeptide of claim 30; and
(b) one or more further polypeptides comprising an amino acid sequence selected from the group consisting of:
(A) an amino acid sequence at least 75% identical to the amino acid sequence of SEQ ID NO:35 residues 1-378, wherein
(i) the polypeptide degrades a PQPQLP (SEQ ID NO:34) peptide at pH 4; and
(ii) residue 278 is Ser, residue 78 is Glu, and residue 82 is Asp
(B) an amino acid sequence at least 75% identical to the amino acid sequence of SEQ ID NO:1 residues 1-567, wherein
(i) the polypeptide degrades a PQPQLP (SEQ ID NO:34) peptide at pH 4; and
(ii) residue 467 is Ser, residue 267 is Glu, and residue 271 is Asp, wherein the polypeptides are capable of hydrolyzing gliadin.

37. The composition of claim 36, wherein at least one of the one or more polypeptides comprises an amino acid sequence selected from the group consisting of SEQ ID NO: 74, SEQ ID NO: 75, SEQ ID NO: 77, SEQ ID NO: 78, SEQ ID NO: 88, SEQ ID NO: 89, SEQ ID NO: 98, and SEQ ID NO: 111, or an analog thereof.

38. The composition of claim 36, wherein at least one of the one or more further polypeptides comprises the amino acid sequence of SEQ ID NO: 1 residues 1-567 and/or SEQ ID NO: 35 residues 1-378.

39. The composition of claim 36, wherein at least one of the one or more further polypeptides comprises an amino acid sequence selected from the group consisting of SEQ ID NO: 2-33 residues 1-567 and 36-67 residues 1-378.

40. The composition of claim 36, wherein at least one of the one or more further polypeptides comprises the amino acid sequence of SEQ ID NO: 90, or an analog thereof.

41. A pharmaceutical composition comprising the isolated polypeptide of claim 30 and a pharmaceutically acceptable carrier.

42. A pharmaceutical composition comprising the composition of claim 36, and a pharmaceutically acceptable carrier.

43. A method for treating celiac sprue, comprising administering to a subject with celiac sprue an amount effective to treat the celiac sprue the isolated polypeptide of claim 30.

44. A method for treating celiac sprue, comprising administering to a subject with celiac sprue an amount effective of the pharmaceutical composition of claim 42 to treat the celiac sprue.

45. The method of claim 43 wherein the polypeptide is administered orally.

46. The method of claim 44 wherein the polypeptide is administered orally.


SEQUENCE LISTING (112 sequences)

SEQ ID NO: 1 - protein, 573 aa, synthetic construct
  REGION 1..573: note = Synthetic
  SITE 119: note = MISC_FEATURE - X can be V or D
  SITE 262: note = MISC_FEATURE - X can be S, K or G
  SITE 291: note = MISC_FEATURE - X can be N or D
  SITE 292: note = MISC_FEATURE - X can be T or S
  SITE 293: note = MISC_FEATURE - X can be D, A, T, or N
  SITE 319: note = MISC_FEATURE - X can be G or S
  SITE 354: note = MISC_FEATURE - X can be S or N
  SITE 357: note = MISC_FEATURE - X can be T or A
  SITE 358: note = MISC_FEATURE - X can be D, N or G
  SITE 361: note = MISC_FEATURE - X can be Q or D
  SITE 368: note = MISC_FEATURE - X can be D, S, or H
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPXDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPXGPDGEVELDIEVAGALAPGAKFAVYFAPXXXAGFLDAITTAIHDPTLKPSVVSISWXGPEDSWTSAAIAAMNRAFLDAAALGVTVLAAAGDXGSXXGEXDGLYHVXFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 2 - protein, 573 aa, synthetic construct
  REGION 1..573: note = Synthetic
  SITE 119: note = MISC_FEATURE - X can be V or D
  SITE 262: note = MISC_FEATURE - X can be S, K or G
  SITE 291: note = MISC_FEATURE - All mutants with more than 10-fold activity have this substitution
  SITE 292: note = MISC_FEATURE - X can be T or S
  SITE 293: note = MISC_FEATURE - X can be D, A, T or N
  SITE 319: note = MISC_FEATURE - X can be G or S
  SITE 354: note = MISC_FEATURE - X can be S or N
  SITE 357: note = MISC_FEATURE - X can be T or A
  SITE 358: note = MISC_FEATURE - X can be D, N or G
  SITE 361: note = MISC_FEATURE - X can be Q or D
  SITE 368: note = MISC_FEATURE - X can be D, S or H
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPXDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPXGPDGEVELDIEVAGALAPGAKFAVYFAPDXXAGFLDAITTAIHDPTLKPSVVSISWXGPEDSWTSAAIAAMNRAFLDAAALGVTVLAAAGDXGSXXGEXDGLYHVXFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 3 - protein, 573 aa, synthetic construct; identical to SEQ ID NO: 2
  REGION 1..573: note = Synthetic
  SITE 119: note = MISC_FEATURE - X can be V or D
  SITE 262: note = MISC_FEATURE - X can be S, K, or G
  SITE 291: note = MISC_FEATURE - All mutants with more than 20-fold activity increase have this substitution together with 358 substitution
  SITE 292: note = MISC_FEATURE - X is T or S
  SITE 293: note = MISC_FEATURE - X is D, A, T or N
  SITE 319: note = MISC_FEATURE - X is G or S
  SITE 354: note = MISC_FEATURE - X is S or N
  SITE 357: note = MISC_FEATURE - X is T or A
  SITE 358: note = MISC_FEATURE - X is N or G (most have G at this position)
  SITE 361: note = MISC_FEATURE - X is Q or D
  SITE 368: note = MISC_FEATURE - X is D, S, or H

SEQ ID NO: 4 - protein, 573 aa, synthetic construct
  REGION 1..573: note = Synthetic
  SITE 119: note = MISC_FEATURE - X is V or D
  SITE 262: note = MISC_FEATURE - X is S, K or G
  SITE 291: note = MISC_FEATURE - All mutants with more than 50-fold activity increase have this substitution together with 319, 358, and 368 substitutions
  SITE 292: note = MISC_FEATURE - X is T or S
  SITE 293: note = MISC_FEATURE - X is D, A, T, or N
  SITE 354: note = MISC_FEATURE - X is S or N
  SITE 357: note = MISC_FEATURE - X is T or A
  SITE 361: note = MISC_FEATURE - X is Q or D
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPXDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPXGPDGEVELDIEVAGALAPGAKFAVYFAPDXXAGFLDAITTAIHDPTLKPSVVSISWSGPEDSWTSAAIAAMNRAFLDAAALGVTVLAAAGDXGSXGGEXDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 5 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWGGPEDSWTSAAIAAMNRAFLDAAALGVTVLAAAGDSGSTNGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 6 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWGGPEDSWTSAAIAAMNRAFLDAAALGVTVLAAAGDSGSTDGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 7 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWGGPEDSWTSAAIAAMNRAFLDAAALGVTVLAAAGDSGSTGGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 8 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWGGPEDSWTSAAIAAMNRAFLDAAALGVTVLAAAGDSGSADGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 9 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTAAGFLDAITTAIHDPTLKPSVVSISWGGPEDSWTSAAIAAMNRAFLDAAALGVTVLAAAGDSGSTDGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 10 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWSGPEDSWTSAAIAAMNRAFLDAAALGVTVLAAAGDSGSTDGEQDGLYHVSFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 11 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWGGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 12 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWGGPEDSWTSAAIAAMNRAFLDAAALGVTVLAAAGDSGSTDGEDDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 13 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWGGPEDSWTSAAIAAMNRAFLDAAALGVTVLAAAGDSGSTNGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 14 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTAAGFLDAITTAIHDPTLKPSVVSISWGGPEDSWTSAAIAAMNRAFLDAAALGVTVLAAAGDSGSTDGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 15 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTAAGFLDAITTAIHDPTLKPSVVSISWGGPEDSWTSAAIAAMNRAFLDAAALGVTVLAAAGDSGSTNGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 16 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 17 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWGGPEDSWTSAAIAAMNRAFLDAAALGVTVLAAAGDNGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 18 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWGGPEDSWTSAAIAAMNRAFLDAAALGVTVLAAAGDSGSTGGEDDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 19 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 20 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTAAGFLDAITTAIHDPTLKPSVVSISWGGPEDSWTSAAIAAMNRAFLDAAALGVTVLAAAGDSGSTNGEDDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 21 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWSGPEDSWTSAAIAAMNRAFLDAAALGVTVLAAAGDNGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 22 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTAAGFLDAITTAIHDPTLKPSVVSISWGGPEDSWTSAAIAAMNRAFLDAAALGVTVLAAAGDSGSTGGEDDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 23 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWSGPEDSWTSAAIAAMNRAFLDAAALGVTVLAAAGDSGSTGGEDDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 24 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWSGPEDSWTSAAIAAMNRAFLDAAALGVTVLAAAGDNGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 25 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTAAGFLDAITTAIHDPTLKPSVVSISWSGPEDSWTSAAIAAMNRAFLDAAALGVTVLAAAGDSGSTGGEDDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 26 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWSGPEDSWTSAAIAAMNRAFLDAAALGVTVLAAAGDNGSTGGEDDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 27 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTAAGFLDAITTAIHDPTLKPSVVSISWSGPEDSWTSAAIAAMNRAFLDAAALGVTVLAAAGDNGSTGGEDDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 28 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDSDAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 29 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTTAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 30 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPGGPDGEVELDIEVAGALAPGAKFAVYFAPDSDAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 31 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPKGPDGEVELDIEVAGALAPGAKFAVYFAPDTNAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 32 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPDDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPKGPDGEVELDIEVAGALAPGAKFAVYFAPDTTAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 33 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWGGPEDSWTSAAIAAMNRAFLDAAALGVTVLAAAGDSGSTDGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 34 - protein, 6 aa, synthetic construct
  REGION 1..6: note = Synthetic
PQPQLP

SEQ ID NO: 35 - protein, 384 aa, synthetic construct
  REGION 1..384: note = Synthetic
  SITE 73: note = MISC_FEATURE - X can be S, K or G
  SITE 102: note = MISC_FEATURE - X can be N or D
  SITE 103: note = MISC_FEATURE - X can be T or S
  SITE 104: note = MISC_FEATURE - X can be T or S
  SITE 130: note = MISC_FEATURE - X can be G or S
  SITE 165: note = MISC_FEATURE - X can be S or N
  SITE 168: note = MISC_FEATURE - X can be T or A
  SITE 169: note = MISC_FEATURE - X can be D, N or G
  SITE 172: note = MISC_FEATURE - X can be Q or D
  SITE 179: note = MISC_FEATURE - X can be D, S, or H
AAPTAYTPLDVAQAYQFPEGLDGQGQCIAIIELGGGYDEASLAQYFASLGVPAPQVVSVSVDGASNQPTGDPXGPDGEVELDIEVAGALAPGAKFAVYFAPXXXAGFLDAITTAIHDPTLKPSVVSISWXGPEDSWTSAAIAAMNRAFLDAAALGVTVLAAAGDXGSXXGEXDGLYHVXFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 36 - protein, 384 aa, synthetic construct
  REGION 1..384: note = Synthetic
  SITE 73: note = MISC_FEATURE - X can be S, K or G
  SITE 102: note = MISC_FEATURE - All mutants with more than 10-fold activity have this substitution
  SITE 103: note = MISC_FEATURE - X can be T or S
  SITE 104: note = MISC_FEATURE - X can be D, A, T or N
  SITE 130: note = MISC_FEATURE - X can be G or S
  SITE 165: note = MISC_FEATURE - X can be S or N
  SITE 168: note = MISC_FEATURE - X can be T or A
  SITE 169: note = MISC_FEATURE - X can be D, N, or G
  SITE 172: note = MISC_FEATURE - X can be Q or D
  SITE 179: note = MISC_FEATURE - X can be D, S, or H
AAPTAYTPLDVAQAYQFPEGLDGQGQCIAIIELGGGYDEASLAQYFASLGVPAPQVVSVSVDGASNQPTGDPXGPDGEVELDIEVAGALAPGAKFAVYFAPDXXAGFLDAITTAIHDPTLKPSVVSISWXGPEDSWTSAAIAAMNRAFLDAAALGVTVLAAAGDXGSXXGEXDGLYHVXFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 37 - protein, 384 aa, synthetic construct; identical to SEQ ID NO: 36
  REGION 1..384: note = Synthetic
  SITE 73: note = MISC_FEATURE - X is S, K, or G
  SITE 102: note = MISC_FEATURE - All mutants with more than 20-fold activity increase have this substitution together with 358 substitution
  SITE 103: note = MISC_FEATURE - X is T or S
  SITE 104: note = MISC_FEATURE - X is D, A, T or N
  SITE 130: note = MISC_FEATURE - X is G or S
  SITE 165: note = MISC_FEATURE - X is S or N
  SITE 168: note = MISC_FEATURE - X is T or A
  SITE 169: note = MISC_FEATURE - X is N or G (most have G at this position)
  SITE 172: note = MISC_FEATURE - X is Q or D
  SITE 179: note = MISC_FEATURE - X is D, S, or H

SEQ ID NO: 38 - protein, 384 aa, synthetic construct
  REGION 1..384: note = Synthetic
  SITE 73: note = MISC_FEATURE - X is S, K, or G
  SITE 102: note = MISC_FEATURE - All mutants with more than 50-fold activity increase have this substitution together with 319, 358, and 368 substitutions
  SITE 103: note = MISC_FEATURE - X is T or S
  SITE 104: note = MISC_FEATURE - X is D, A, T, or N
  SITE 165: note = MISC_FEATURE - X is S or N
  SITE 168: note = MISC_FEATURE - X is T or A
  SITE 172: note = MISC_FEATURE - X is Q or D
AAPTAYTPLDVAQAYQFPEGLDGQGQCIAIIELGGGYDEASLAQYFASLGVPAPQVVSVSVDGASNQPTGDPXGPDGEVELDIEVAGALAPGAKFAVYFAPDXXAGFLDAITTAIHDPTLKPSVVSISWSGPEDSWTSAAIAAMNRAFLDAAALGVTVLAAAGDXGSXGGEXDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 39 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWGGPEDSWTSAAIAAMNRAFLDAAALGVTVLAAAGDSGSTNGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 40 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWGGPEDSWTSAAIAAMNRAFLDAAALGVTVLAAAGDSGSTDGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 41 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWGGPEDSWTSAAIAAMNRAFLDAAALGVTVLAAAGDSGSTGGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 42 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWGGPEDSWTSAAIAAMNRAFLDAAALGVTVLAAAGDSGSADGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 43 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTAAGFLDAITTAIHDPTLKPSVVSISWGGPEDSWTSAAIAAMNRAFLDAAALGVTVLAAAGDSGSTDGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 44 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWSGPEDSWTSAAIAAMNRAFLDAAALGVTVLAAAGDSGSTDGEQDGLYHVSFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 45 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWGGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 46 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWGGPEDSWTSAAIAAMNRAFLDAAALGVTVLAAAGDSGSTDGEDDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 47 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWGGPEDSWTSAAIAAMNRAFLDAAALGVTVLAAAGDSGSTNGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 48 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTAAGFLDAITTAIHDPTLKPSVVSISWGGPEDSWTSAAIAAMNRAFLDAAALGVTVLAAAGDSGSTDGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 49 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTAAGFLDAITTAIHDPTLKPSVVSISWGGPEDSWTSAAIAAMNRAFLDAAALGVTVLAAAGDSGSTNGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 50 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 51 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWGGPEDSWTSAAIAAMNRAFLDAAALGVTVLAAAGDNGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 52 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWGGPEDSWTSAAIAAMNRAFLDAAALGVTVLAAAGDSGSTGGEDDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 53 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 54 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTAAGFLDAITTAIHDPTLKPSVVSISWGGPEDSWTSAAIAAMNRAFLDAAALGVTVLAAAGDSGSTNGEDDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 55 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWSGPEDSWTSAAIAAMNRAFLDAAALGVTVLAAAGDNGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 56 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTAAGFLDAITTAIHDPTLKPSVVSISWGGPEDSWTSAAIAAMNRAFLDAAALGVTVLAAAGDSGSTGGEDDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 57 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWSGPEDSWTSAAIAAMNRAFLDAAALGVTVLAAAGDSGSTGGEDDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 58 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWSGPEDSWTSAAIAAMNRAFLDAAALGVTVLAAAGDNGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 59 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTAAGFLDAITTAIHDPTLKPSVVSISWSGPEDSWTSAAIAAMNRAFLDAAALGVTVLAAAGDSGSTGGEDDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 60 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWSGPEDSWTSAAIAAMNRAFLDAAALGVTVLAAAGDNGSTGGEDDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 61 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTAAGFLDAITTAIHDPTLKPSVVSISWSGPEDSWTSAAIAAMNRAFLDAAALGVTVLAAAGDNGSTGGEDDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 62 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDSDAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 63 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTTAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 64 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPGGPDGEVELDIEVAGALAPGAKFAVYFAPDSDAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 65 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPKGPDGEVELDIEVAGALAPGAKFAVYFAPDTNAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 66 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPKGPDGEVELDIEVAGALAPGAKFAVYFAPDTTAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 67 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWGGPEDSWTSAAIAAMNRAFLDAAALGVTVLAAAGDSGSTDGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 68 - protein, 4 aa, synthetic construct
  REGION 1..4: note = Synthetic
PQLP

SEQ ID NO: 69 - protein, 6 aa, synthetic construct
  REGION 1..6: note = Synthetic
  SITE 1: note = MISC_FEATURE - N-terminal QXL520
  SITE 6: note = MISC_FEATURE - C-terminal K(5-FAM)
PQPQLP

SEQ ID NO: 70 - protein, 12 aa, synthetic construct
  REGION 1..12: note = Synthetic
QLQPFPQPQLPY

SEQ ID NO: 71 - protein, 290 aa, synthetic construct
  REGION 1..290: note = Synthetic
MVRVPVPQLQPQNPSQQQPQEQVPLVQQQQFPGQQQPFPPQQPYPQPQPFPSQQFYLQLQPFPQPQLPYPQPQLPYPQPQLPYPQPQPFRPQQPYPQSQPQYSQPQQPISQQQQQQQQQQQQKQQQQQQQQILQQILQQQLIPCRDVVLQQHSIAYGSSQVLQQSTYGLVQQLCCQQLWQIPEQSRCQAIHNVVHAIILHQQQQQQQQQQQQPLSQVSFQQPQQQYPSGQGSFQPSQQNPQAQGSVQPQQLPQFEEIRNLALETLPAMCNVYIPPYCTIAPVGIFGTNYR

SEQ ID NO: 72 - protein, 33 aa, synthetic construct
  REGION 1..33: note = Synthetic
LQLQPFPQPQLPYPQPQLPYPQPQLPYPQPQPF

SEQ ID NO: 73 - protein, 26 aa, synthetic construct
  REGION 1..26: note = Synthetic
FLQPQQPFPQQPQQPYPQQPQQPFPQ

SEQ ID NO: 74 - protein, 512 aa, synthetic construct
  REGION 1..512: note = Synthetic
MAPSDVEIVDPVAPEERITVTVLLRRRSSIPDQLIEGPDTLSRAELADRHGADPADVEAVRVAMSGAGLTVVGTDLPSRRVTVAGTAEALMRTFGAELQIVRDASGFQHRARSGELRIPAALDGIVIAVLGLDNRPQAEARFRASQPEAARSFRPDALGRVYRFPANTDGTGQTIAIVELGGGFRQSELDTYFGGLGIPAPQVLAVGVDGGQNLPSGDAGSADGEVLLDIEVAGALAPGARQVVYFAPNTDRGFVDAVTTAVHADPTPAAVSISWGAPEDKWTAQARRAFDAALADAAALGVTVTAAAGDRGSADGEGGGGLHTDFPASSPHLLACGGTKLAVADGGTVASETVWNGGERGGATGGGVSVAFGLPAYQRNAGVDKRRKTGKPGRGVPDVAAVADPATGYEVLVDGEQLVFGGTSAVAPLWAALVARLTQALGRPLGLLNTALYDGAQPGRTQPGFRDVTEGDNDISGKHGPYPARAGWDACTGLGVPDGEALLAALRKPGKE

SEQ ID NO: 75 - protein, 523 aa, synthetic construct
  REGION 1..523: note = Synthetic
  SITE 106: note = MISC_FEATURE - Xaa is V or D
  SITE 246: note = MISC_FEATURE - Xaa is N, S, K or G
  SITE 275: note = MISC_FEATURE - Xaa is N or D
  SITE 276: note = MISC_FEATURE - Xaa is T or S
  SITE 277: note = MISC_FEATURE - Xaa is D, A, T or N
  SITE 303: note = MISC_FEATURE - Xaa is G or S
  SITE 342: note = MISC_FEATURE - Xaa is D, G or N
  SITE 352: note = MISC_FEATURE - Xaa is D, S or H
MANHPLNGSERECLKDAQPIGKADPNERLEVTMLVRRRSHDAFEKHISALAAQGASAKHIDHDEFTKHFGADSADLAAVHAFAQKHGLSVVESHEARRAVVLSGTXAQFDAAFGVSLQQYEHDGGTYRGRTGPIHLPDELNGVVDAVMGLDNRPQARPSFRTRAQGNVRWTARAAGASTFTPVQLASLYDFPQGDGQNQCIGIIELGGGYRPADLKTYFASLNMKAPSVTAVSVDHGRNHPTGDPXGPDGEVMLDIEVAGAVAPGAKIVVYFAPXXXAGFIDAIGTAIHDTKNKPSVISISWXGPESAWTQQAMNAFDQAFQSAAALGVTICAASGDNGSGXGVGDGADHVXFPASSPYALGCGGTSLQASGNGIASETVWNDGANGGATGGGVSSFFALPAWQEGLRVTRAGGAHSPLAMRGVPDVAGNADPVTGYEVRVDGHDMVIGGTSAVAPLWAGLIARINAIKGAPVGYINPHLYKDPLALVDITKGNNDDFHATAGWDACTGLGRPDGKKVKDAVS

SEQ ID NO: 76 - protein, 554 aa, synthetic construct
  REGION 1..554: note = Synthetic
  SITE 120: note = MISC_FEATURE - Xaa is V or D
  SITE 259: note = MISC_FEATURE - Xaa is N, S, K or G
  SITE 288: note = MISC_FEATURE - Xaa is N or D
  SITE 289: note = MISC_FEATURE - Xaa is S or T
  SITE 290: note = MISC_FEATURE - Xaa is D, A, T, or N
  SITE 316: note = MISC_FEATURE - Xaa is G or S
  SITE 351: note = MISC_FEATURE - Xaa is D, S or N
  SITE 354: note = MISC_FEATURE - Xaa is T or A
  SITE 355: note = MISC_FEATURE - Xaa is D, N, or G
  SITE 358: note = MISC_FEATURE - Xaa is Q or D
  SITE 365: note = MISC_FEATURE - Xaa is D, S or H
MNHDHSPTGGELSNWVRVPGSERAAVQGSRKVGPADPNEQMSVTVVVRRPAADTAVTSMIEKVGAQPLSERRHLTREEFASTHGANPADLSKVEKFAHEHNLQVKEVNAAAGTMVLSGTXTSFSKAFGVELSTYEHPDFTYRGRIGHVHIPDYLADTIQSVLGLDNRPQASPRFRVLKEEGGVTTAHAGRTSYTPLEVAALYNFPSIHCKDQCIGILELGGGYRPADLQTYFNGLGIPQPNITDVSVGGAANRPTGDPXGPDGEVVLDIEVAAAVTPGAKIAVYFADXXXDGFLNAITTAIHDTRNKPSVISISWXKAEIGWTPQAINAMNQAFRDAAALGVTICCASGDXGSXXRVXDGRYHVXFPASSPYVLACGGTRLESSGSTITQEVVWNEGALGGGATGGGVSDVFDRPNWQANANVPTSANPERRIGRGVPDWAGNADPATGYQILVDGTRAVIGGTSAVAPLFAGLIAIINQKLGHSVGFINPILYNLSAQHNVFHDITSGNNDMSGQNGPYEAQPGWDACTGLGSPDGTKLMNAISEAHRLVSVG

SEQ ID NO: 77 - protein, 507 aa, synthetic construct
  REGION 1..507: note = Synthetic
MAPEERRTLPGSAMPRPAGAQVLGQIPDDERVEVTVVLQPRAPLPEPGPTPMSRAELADLRSPPEGALEAIARYVAGQGLEVIAADAPRRRIVLAGSAARIAALFGISFVRLQLEGRRYRTYEGEISLPAELAPLVVAVLGLDTRPFARSHRRPAVAPNAPTTAPTVARAYDFPTAYDGRGTTIGFIELGGGFQESDLVRYCEGLGLSTPQVSVVGVDGARNAPTGDPNGPDAEVMLDLEVATGVANGADLVLYMAANTDAAFYSAIATALRDATHAPVAISISWGAPEESYPATTIAAFESVLEEAVHVGVTVLVAAGDQGSTDGVDDGRAHVDYPAASPYVLACGGTRLDLDGTTIVAETVWNDLPNGGATGGGISALFPVPSWQAGIAMPPSANPGAGPGRGVPDVAGNADPDTGYRIVVDGVATVVGGTSAVAPLWAGLVARCHQAGARGGFWNPLLYAARGSSAFHEITVGSNGAYDAGPIWNACCGLGSPNGTAILQTLRA

SEQ ID NO: 78 - protein, 507 aa, synthetic construct
  REGION 1..507: note = Synthetic
MAPEERRTLPGSAMPRPAGAQVLGQIPDDERVEVTVVLQPRAPLPEPGPTPMSRAELADLRSPPEGALEAIARYVAGQGLEVIAADAPRRRIVLAGSAARIAALFGISFVRLQLEGRRYRTYEGEISLPAELAPLVVAVLGLDTRPFARSHRRPAVAPNAPTTAPTVARAYDFPTAYDGRGTTIGFIELGGGFQESDLVRYCEGLGLSTPQVSVVGVDGARNAPTGDPNGPDAEVMLDLEVATGVANGADLVLYMAANTDAAFYSAIATALRDATHAPVAISISWSAPEESYPATTIAAFESVLEEAVHVGVTVLVAAGDQGSTGGVDDGRAHVHYPAASPYVLACGGTRLDLDGTTIVAETVWNDLPNGGATGGGISALFPVPSWQAGIAMPPSANPGAGPGRGVPDVAGNADPDTGYRIVVDGVATVVGGTSAVAPLWAGLVARCHQAGARGGFWNPLLYAARGSSAFHEITVGSNGAYDAGPIWNACCGLGSPNGTAILQTLRA

SEQ ID NO: 79 - protein, 532 aa, synthetic construct
  REGION 1..532: note = Synthetic
MTKQPVSGSSDKIHPDDAKCIGDCDPSEQIEVIVMLRRKDEAGFRQMMSRIDAGEAPGQAVSREEFDRRFTASDEDIDKVKAFAKQYGLSVERAETETRSVVLKGTIEQFQKAFDVKLERFQHHNIGEYRGRTGPVNVPDEMHDAVTAVLGLDSKPQARPHFRFRPPFKPLRGAAPASFSPVDLAKLYDFPDGDGAGQCIAIIELGGGYRDSDLSAYFSKLGVKAPTVVPVGVDGGKNAPTGNPNGPDGEVTLDIEIAGAIAPGARIAVYFAPNSDAGFVDAVNRALHDAANKPSVISISWGGPESNWSPQSMSAFNDVLQSAAALGVTVCAASGDGGSADGVGDGADHVDFPASSPYVLGCGGTSLAASGAGIAKEVVWNDGDQGGAGGGGVSGTFALPVWQKGLSVTRNGKHIALAKRGVPDVAGDASPQTGYEVLIDGEDTVVGGTSAVAPLWAALIARINAIDASPAGFVNPKLYKAKTAFRDITEGNNGSFSAAAGWDACTGMGSPDGGKIAAALKPAKPSQSAGQQ

SEQ ID NO: 80 - protein, 544 aa, synthetic construct
  REGION 1..544: note = Synthetic
MGRLQGSYRPSLGTPVGPVPDDQPIDVTVVLRPTAADDFRADPDDVAAVRAFAGRAGLDVAEVDEPARTVRLRGPAAAARTAFDTPLALYDSGGRAIRGREGDLGLPDELDDRVVAVLGLDERPAARPRFQPAASARQGLTALQVARAYDFPAATGEGQTIAIIELGGGFGQADLDTYFGGLDLPTPAVSAVGVQGAANVPGGDPDGADGEVLLDIEVAGAVAPGAAQVVYFAPNTDAGFLAAINAAAAATPRPAAISISWGGPESSWTAQAMRAYDQAFAAARAAGITVLAAAGDAGADDATDRLVADFPAGSPNVIACGGTKLTLDAAGARASEVVWNEAADSATGGGYSATFTRPAWQPAAVGRYRGLPDISGNADPQTGYRVVVDGQPTVVGGTSAVAPLLAGLVARLAQLTGAPVADLAAVAYANPAAFTDITAGDNQGYPARSGWDPASGLGSPVGTKLLTAVGGPTPPPTTPPPTTPPPTTPPPTIPPPTTPPTQTVDAADRALWSAVATWAGGTHTGANARAAKAVRAWAQAKSLA

SEQ ID NO: 81 - protein, 523 aa, synthetic construct
  REGION 1..523: note = Synthetic
MTQPRYTPLPGSEREAPLLAARSNATAARASRAQTASATVVLRRRSELPEALVLDQQFISSDELAARYGADPVDIEKVRSVLERFKVSVVEVDAASRRVKVEGAVADIERAFNIALHSASGTDPHSGRGFEYRYRTGVLSVPAELGGIVTAVLGLDNRRQAETRLRVVPAAALGSSYTPVQLGEIYNFPQDATGAGQRIAIIELGGGYTPAGLRRYFASLGVVPPKVAAVSVDGAQNAPGPDPGADGEVQLDVEVAGALAPGAHVLVYFAPNTDQGFLDAVSQAAHATPPPTAISISWGASEDSWTASARDALNQALRDAAALGVTVTAAAGDSGSSDGVPDRRAHVDFPASSPYVLATGGTSLRADPATGVVQSETVWNDSQGSTGGGVSDVFPRPAWQAHVDVPHAGRGVPDVSAVADPATGYQVLVDNQPAVIGGTSAVAPLWAALVARLAESLGRPLGLLQPLVYPRTPGSTAYPGFRDITIGNNGAYKAGKGWDAATGLGVPDGTELLAHLRGLNGSE

SEQ ID NO: 82 - protein, 537 aa, synthetic construct
  REGION 1..537: note = Synthetic
  SITE 106: note = MISC_FEATURE - Xaa is I, V or D
  SITE 244: note = MISC_FEATURE - Xaa is N, S, K, or G
  SITE 273: note = MISC_FEATURE - Xaa is N or D
  SITE 274: note = MISC_FEATURE - Xaa is S or T
  SITE 275: note = MISC_FEATURE - Xaa is D, A, T or N
  SITE 301: note = MISC_FEATURE - Xaa is G or S
  SITE 340: note = MISC_FEATURE - Xaa is D, N, or G
  SITE 350: note = MISC_FEATURE - Xaa is D, S, or H
MARHLHAGSEPKVITESKCIGACDPAERIHVTVMLRREGEQALDALVDKLASGDPAAKPVSREDFAKRFGARADDIQHTEAFAKRHQLTVERVDPVQSVVELAGTXAQFENAFGVKLEKYEHHAIGSFRARTGAIALPDELHDAVTAVLGLDTRPQAHPHFRFRPPFQPARSGAGTSYTPLQLASIYNFPEGDGAGQCIALVELGGGYRAADIRQYFEQLGVKPPKLVDVSVNGGRNAPTDDPXGPDGEVALDIEVAGAIAPGATIAVYFAGXXXAGFIQSVNQAIHDSTNRPSVVSISWXGPEASWTQQSITAFNNVLKTAASLGVTVCAASGDSGSSXGLQDGSNHVXFPASSPYVLACGGTTLDAQAGQGIRREVVWNDEAASGGAGGGGVSAVFPAPSYQKGLSAKATGGGSTPLSQRGVPDVAGDASPTTGYIISIAGTTAVLGGTSAVAPLWAALIARINANGKSPVGWANPKLYAQPGAFHDITQGNNGAFAASEGWDACTGLGSPDGAKVAAALQGASGGSQQGRATGA

SEQ ID NO: 83 - protein, 519 aa, synthetic construct
  REGION 1..519: note = Synthetic
MTKHPLPGSERVLAPGSKVVAQCDPSETIEVVVVLRRKNEQQFAQMMKTIEAGAAGARPLTREELEQRFGALPEDIAKLKAFAAQHGLSVVREDASARTVVLSGRIEQFQQAFDVQLQHYEHQSMGRFRGRTGAISVPDELHGVVTAVLGLDDRPQARPHFRIRPPFQPARAQSASSFTPLQLASLYRFPQGDGSGQCIGIVELGGGYRTADLDSYFSSLGVGSPKVVAVGVDQSGNQPTGDPNGPDGEVTLDIEIAGALAPAATIAVYFTTNSDAGFIDAVSQAVHDRTNQPSVISISWGAPESMWTAQSMKALNDVLQSAAAIGVTVCAASGDSGSSDGVGDGRDHVDFPASSPYVLACGGTSLQGSGRTVAHEVVWNDGSNGGATGGGVSGAFPVPAWQEGLSTSAAQGGQRALTGRGVPDVAGDASPLTGYDVIVDGNNTVIGGTSAVAPLWAALIARINGAKGAPVGFVNPKLYKASACNDITQGNNGSYAATTGWDACTGLGSPDGVKVAAAL

SEQ ID NO: 84 - protein, 512 aa, synthetic construct
  REGION 1..512: note = Synthetic
MSPIASRRSALPLSERPAPENARALAAVEPDRTMTVSVLVRRKKPLVLADLEGKKLTHREFERRYGASEKDFATIAKFAAGHGLAVDHHASSLARRTVVLRGTARQMQQAFGVTLHDYEDSETQQRYHSFTGAITVPAAHARIIESVLGLDARPIAKPHFRVRKRSAAATGAVSFNPPQVASLYSFPTGVDGSGETIGILELGGGYETSDIQQYFSGLGIQPPTVVAVSVDGAVNAPGNPNGADGEVALDIQVAGSIAPGAKLAVYFAPNTEQGFVDAITTAVHDTANKPSVLSISWGGPESSWPQAAAQSLNNACESAAALGVTITVASGDNGSTDGVQDGQNHVDFPASSPYVLACGGTYLAAVNNGVPQESVWDDLASGGGATGGGVSALFPLPAWQTGANVPGGSMRGVPDVAGDASPESGYNVLVDGQPQVVGGTSAVAPLWAALIALVNQQKGEAAGFVNAALYQNPSAFHDITQGSNGAYAAAPGWDPCTGLGSPMGTAIAKILA

SEQ ID NO: 85 - protein, 531 aa, synthetic construct
  REGION 1..531: note = Synthetic
MSAFDQLVPLPGSEKTVPDAAPSQTLDPNEVLTVTIRIRRKRTLASLVSTTAPVTEVVSRSEYASRFGADPAIVKQVEAFASAYDLSLVEQSLARRSVLLRGTVAQMEQAFGVSLANYQLADGTVFRGRTGVVNVPSELVEHIEGVFGLDNRPQARAHFQVYKPEKGTKVAPRAGGISYTPPQLARLYNFPTGVTGKGQCIAIIELGGGFRTADIKTYFGGLGLKPPTVVAVSVDGGHNAPSTADSADGEVMLDIDVAGGVAPGAKIVVYFAPNTDQGFLDAITTAMHDTKNKPSVISISWGAAESNWTPQALTSFNQAFQAAAALGITVCAAAGDTGSDDSVGDGKAHVDFPASSPFVLACGGTKLTATDNVIASEVVWHESKTSATGGGVSDVFDLPDYQQKSHVPPSVNDKTRIGRGVPDVAAVADPVTGYAVRVDGSNLVFGGTSAVAPLMAGLIALINQQRGKAVGFIHPLIYANPSAFRDITQGNNTTTTGNKGYAATTGWDACTGLGVADGKKLASVLTATPVA

SEQ ID NO: 86 - protein, 567 aa, synthetic construct
  REGION 1..567: note = Synthetic
MAATPRFASQPRVTLPGSQKHPLTTDTEVPPPAPVKAAATKLSATPFTVTVIVKRKNPLNLKQVLKPAGRLTHAAFAKAHGPSPDGVKLVKAFAKEFGLTVAPAPGQGRRALYLTGTAAAMQTAFGVTFATKIMEGTKYRVREGDICLPKELIGHVDAVLGLDNRPQAKPHFRHHKPAATSVSYTPVQVGQLYGFPSGAKATGQTIGLIELGGGFRAADITAYFKTLGQTAPKVTAVLVDKAKNTPTTSSSADGEVMLDIEVAAAVAPGANIAVYFAPNTDQGFIDAISQAVHDTVNKPSVISISWGGPESTWTAQSLAALDAACQSAAALGITITVAAGDDGSTDGVKGTVNHVDFPASSPHVLGCGGTKLLGSGTTITSEVVWNELTANEGATGGGVSNVFPLPTWQAKSNVPKPTVAAGGRGVPDVSGNADPSTGYTVRVDGSTFPIGGTSAVAPLWAGLIALCNAQNKTTAGFINPALYAAAAAKSFRDITSGNNGGFKAGPGWDACTGLGSPIGTAIAKTLAPATKSTSKTAVKNAPEIRFRPHKKAPTKTAAKTPALRRLK

SEQ ID NO: 87 - protein, 543 aa, synthetic construct
  REGION 1..543: note = Synthetic
MPTSSRFASQSRVPLPGSERKPFVPAGAPKAAKTPKVSTAVKTVPATGRIRVSLIVPPKQPLDTKRLGKLDARLSRAQFAARHGADPASVRLVKAFAKEFGLTVEPITQPGRCTVQLSGTCAAMRKAFAISLVEHTTEQGKFRLREGEISLPAELEGHVLAVLGLDNRPQAKPHFRIAKPRATNVSYTPVQVAQMYGFPAGATATGQTIGIIELGGGYRAADLTAYFKTLGLPAPTVTAVPIDGGKNTPGNANGADGEVMLDIEVCAAVAQGAKIAVYFTTNTDQGFIDAITTAVHDSTNKPSVISISWGGPESSWTEQSMTALDAACQAAAAVGVTITVAAGDNGSSDGASGDNVDFPASSPHVLACGGTKLVGSGSTITSEVVWDETSNDEGATGGGVSTVFALPTWQKNANVPSPTTSAGGRGVPDVSGDADPSTGYTIRVDSETTVIGGTSAVAPLWAGLIALANAQNKVAAGFVNPALYAAGAKKAFRDITQGNNGSFSAGPGWDACTGLGSPVGNLVIQAVAPKSTTTKKAKKGKTK

SEQ ID NO: 88 - protein, 378 aa, synthetic construct
  REGION 1..378: note = Synthetic
  SITE 111: note = MISC_FEATURE - Xaa is N, S, G or K
  SITE 139: note = MISC_FEATURE - Xaa is N or D
  SITE 140: note = MISC_FEATURE - Xaa is N, T or S
  SITE 141: note = MISC_FEATURE - Xaa is E, T, D, A, or N
  SITE 164: note = MISC_FEATURE - Xaa is G or S
  SITE 203: note = MISC_FEATURE - Xaa is D, N or G
  SITE 211: note = MISC_FEATURE - Xaa is D, S or H
MHSYLKQQSHMQSYLEQENHMRSYLEMRKKPYFDDLANIRPGGLTPAQVCQAYQFAKVQPVRPVKLGIVSLAGQYLSSDMSKAFTGYGLPTPVVSTAGSQVLGDLWSNVEXMMDIEIAGAAWAYATGTAATLLMQFEPXXXTGIPNAINALVAAGCEVISISWXAPANLQTMEAITARKEACKQAAVQNVHVFAASGDESLNXGTNSRTPXDPCCDPNVWGVGGTRLVLQADGSIAQESAWGDGNAADKGGGGGFDSREPLPDYQVGVVHSEHRGSPDSSANADPGTGYAIVANGQWLIGGGTSASAPLTAGYVAAILSTLPGPISQSVLQRKLYTAHKTAFRDILLGSNGAPARPGWEEATGLGSINGPGLAAALQS

SEQ ID NO: 89 - protein, 523 aa, synthetic construct
  REGION 1..523: note = Synthetic
MANHPLNGSERECLKDAQPIGKADPNERLEVTMLVRRRSHDAFEKHISALAAQGASAKHIDHDEFTKHFGADSADLAAVHAFAQKHGLSVVESHEARRAVVLSGTVAQFDAAFGVSLQQYEHDGGTYRGRTGPIHLPDELNGVVDAVMGLDNRPQARPSFRTRAQGNVRWTARAAGASTFTPVQLASLYDFPQGDGQNQCIGIIELGGGYRPADLKTYFASLNMKAPSVTAVSVDHGRNHPTGDPNGPDGEVMLDIEVAGAVAPGAKIVVYFAPNTDAGFIDAIGTAIHDTKNKPSVISISWSGPESAWTQQAMNAFDQAFQSAAALGVTICAASGDNGSGGGVGDGADHVHFPASSPYALGCGGTSLQASGNGIASETVWNDGANGGATGGGVSSFFALPAWQEGLRVTRAGGAHSPLAMRGVPDVAGNADPVTGYEVRVDGHDMVIGGTSAVAPLWAGLIARINAIKGAPVGYINPHLYKDPLALVDITKGNNDDFHATAGWDACTGLGRPDGKKVKDAVS

SEQ ID NO: 90 - protein, 567 aa, synthetic construct
  REGION 1..567: note = Synthetic
  REGION 554..567: note = MISC_FEATURE - Amino acids are optionally absent.
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPDDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPKGPDGEVELDIEVAGALAPGAKFAVYFAPDTTAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALE

SEQ ID NO: 91 - protein, 6 aa, synthetic construct
  REGION 1..6: note = Synthetic
QPQLPY

SEQ ID NO: 92 - protein, 9 aa, synthetic construct
  REGION 1..9: note = Synthetic
IQPQQPAQL

SEQ ID NO: 93 - protein, 14 aa, synthetic construct
  REGION 1..14: note = Synthetic
PQPQLPYSQPQPFR

SEQ ID NO: 94 - protein, 20 aa, synthetic construct
  REGION 1..20: note = Synthetic
LQLQPFPQPQLPYPQPQLPY

SEQ ID NO: 95 - protein, 539 aa, synthetic construct
  REGION 1..539: note = Synthetic
  SITE 116: note = MISC_FEATURE - Xaa is I, V or D
  SITE 255: note = MISC_FEATURE - Xaa is N, S, K, or G
  SITE 284: note = MISC_FEATURE - Xaa is N or D
  SITE 285: note = MISC_FEATURE - Xaa is S or T
  SITE 286: note = MISC_FEATURE - Xaa is D, A, T, or N
  SITE 312: note = MISC_FEATURE - Xaa is G or S
  SITE 347: note = MISC_FEATURE - Xaa is S or N
  SITE 350: note = MISC_FEATURE - Xaa is G, T or A
  SITE 351: note = MISC_FEATURE - Xaa is D, N or G
  SITE 354: note = MISC_FEATURE - Xaa is Q or D
  SITE 361: note = MISC_FEATURE - Xaa is D, S or H
MQRGTKEGLNMARHLQADREPRIVPESKCLGQCDPAERIHVTIMLRRQEEGQLDALVHQLATGDARAKPVSRDAFAQRFSANPDDIRKTEDFAHRHQLTVDRVDPVESVVVLSGTXAQFEAAFSVKLERFEHRSIGQYRGRSGPIVLPDDIGDAVTAVLGLDSRPQARPHFRFRPPFKPARGAAAVTFTPIQLASLYDFPAGDGAGQCIAIIELGGGYRAADIQQYFRGLGITTPPKLVDVNVGTGRNAPTGEPXGPDGEVALDIEIAGAIAPAAKIAVYFAPXXXAGFIQAVNAAVTDKTNQPSVISISWXGPEAIWQAQSAQAFNRVLQAAAAQGITVCAASGDXGSXXGLXDGADHVXFPASSPYVLGCGGTQLDALPGQGIRSEVTWNDEASGGGAGGGGVSALFDLPAWQQGLKVARADGTTTPLAKRGVPDVAGDASPQTGYEVSVAGTPAVMGGTSAVAPLWAALIARINAANGASAGWINPVLYKHPGALRDITKGSNGTYAAASGWDACTGLGSPNGAQLATILARKPSS

SEQ ID NO: 96 - protein, 512 aa, synthetic construct; identical to SEQ ID NO: 74
  REGION 1..512: note = Synthetic

SEQ ID NO: 97 - protein, 539 aa, synthetic construct
  REGION 1..539: note = Synthetic
MQRGTKEGLNMARHLQADREPRIVPESKCLGQCDPAERIHVTIMLRRQEEGQLDALVHQLATGDARAKPVSRDAFAQRFSANPDDIRKTEDFAHRHQLTVDRVDPVESVVVLSGTIAQFEAAFSVKLERFEHRSIGQYRGRSGPIVLPDDIGDAVTAVLGLDSRPQARPHFRFRPPFKPARGAAAVTFTPIQLASLYDFPAGDGAGQCIAIIELGGGYRAADIQQYFRGLGITTPPKLVDVNVGTGRNAPTGEPNGPDGEVALDIEIAGAIAPAAKIAVYFAPNSDAGFIQAVNAAVTDKTNQPSVISISWGGPEAIWQAQSAQAFNRVLQAAAAQGITVCAASGDSGSGDGLQDGADHVDFPASSPYVLGCGGTQLDALPGQGIRSEVTWNDEASGGGAGGGGVSALFDLPAWQQGLKVARADGTTTPLAKRGVPDVAGDASPQTGYEVSVAGTPAVMGGTSAVAPLWAALIARINAANGASAGWINPVLYKHPGALRDITKGSNGTYAAASGWDACTGLGSPNGAQLATILARKPSS

SEQ ID NO: 98 - protein, 523 aa, synthetic construct
  REGION 1..523: note = Synthetic
MANHPLNGSERECLKDAQPIGKADPNERLEVTMLVRRRSHDAFEKHISALAAQGASAKHIDHDEFTKHFGADSADLAAVHAFAQKHGLSVVESHEARRAVVLSGTVAQFDAAFGVSLQQYEHDGGTYRGRTGPIHLPDELNGVVDAVMGLDNRPQARPSFRTRAQGNVRWTARAAGASTFTPVQLASLYDFPQGDGQNQCIGIIELGGGYRPADLKTYFASLNMKAPSVTAVSVDHGRNHPTGDPNGPDGEVMLDIEVAGAVAPGAKIVVYFAPNTDAGFIDAIGTAIHDTKNKPSVISISWGGPESAWTQQAMNAFDQAFQSAAALGVTICAASGDNGSGDGVGDGADHVDFPASSPYALGCGGTSLQASGNGIASETVWNDGANGGATGGGVSSFFALPAWQEGLRVTRAGGAHSPLAMRGVPDVAGNADPVTGYEVRVDGHDMVIGGTSAVAPLWAGLIARINAIKGAPVGYINPHLYKDPLALVDITKGNNDDFHATAGWDACTGLGRPDGKKVKDAVS

SEQ ID NO: 99 - protein, 367 aa, synthetic construct
  REGION 1..367: note = Synthetic
AGRTSYTPLEVAALYNFPSIHCKDQCIGILELGGGYRPADLQTYFNGLGIPQPNITDVSVGGAANRPTGDPNGPDGEVVLDIEVAAAVTPGAKIAVYFADNSDDGFLNAITTAIHDTRNKPSVISISWGKAEIGWTPQAINAMNQAFRDAAALGVTICCASGDDGSTDRVQDGRYHVDFPASSPYVLACGGTRLESSGSTITQEVVWNEGALGGGATGGGVSDVFDRPNWQANANVPTSANPERRIGRGVPDWAGNADPATGYQILVDGTRAVIGGTSAVAPLFAGLIAIINQKLGHSVGFINPILYNLSAQHNVFHDITSGNNDMSGQNGPYEAQPGWDACTGLGSPDGTKLMNAISEAHRLVSVG

SEQ ID NO: 100 - protein, 507 aa, synthetic construct; identical to SEQ ID NO: 77
  REGION 1..507: note = Synthetic

SEQ ID NO: 101 - protein, 507 aa, synthetic construct; identical to SEQ ID NO: 78
  REGION 1..507: note = Synthetic

SEQ ID NO: 102 - protein, 532 aa, synthetic construct; identical to SEQ ID NO: 79
  REGION 1..532: note = Synthetic

SEQ ID NO: 103 - protein, 544 aa, synthetic construct; identical to SEQ ID NO: 80
  REGION 1..544: note = Synthetic

SEQ ID NO: 104 - protein, 523 aa, synthetic construct; identical to SEQ ID NO: 81
  REGION 1..523: note = Synthetic

SEQ ID NO: 105 - protein, 537 aa, synthetic construct
  REGION 1..537: note = Synthetic
MARHLHAGSEPKVITESKCIGACDPAERIHVTVMLRREGEQALDALVDKLASGDPAAKPVSREDFAKRFGARADDIQHTEAFAKRHQLTVERVDPVQSVVELAGTIAQFENAFGVKLEKYEHHAIGSFRARTGAIALPDELHDAVTAVLGLDTRPQAHPHFRFRPPFQPARSGAGTSYTPLQLASIYNFPEGDGAGQCIALVELGGGYRAADIRQYFEQLGVKPPKLVDVSVNGGRNAPTDDPNGPDGEVALDIEVAGAIAPGATIAVYFAGNSDAGFIQSVNQAIHDSTNRPSVVSISWGGPEASWTQQSITAFNNVLKTAASLGVTVCAASGDSGSSDGLQDGSNHVDFPASSPYVLACGGTTLDAQAGQGIRREVVWNDEAASGGAGGGGVSAVFPAPSYQKGLSAKATGGGSTPLSQRGVPDVAGDASPTTGYIISIAGTTAVLGGTSAVAPLWAALIARINANGKSPVGWANPKLYAQPGAFHDITQGNNGAFAASEGWDACTGLGSPDGAKVAAALQGASGGSQQGRATGA

SEQ ID NO: 106 - protein, 520 aa, synthetic construct
  REGION 1..520: note = Synthetic
HMTKHPLPGSERVLAPGSKVVAQCDPSETIEVVVVLRRKNEQQFAQMMKTIEAGAAGARPLTREELEQRFGALPEDIAKLKAFAAQHGLSVVREDASARTVVLSGRIEQFQQAFDVQLQHYEHQSMGRFRGRTGAISVPDELHGVVTAVLGLDDRPQARPHFRIRPPFQPARAQSASSFTPLQLASLYRFPQGDGSGQCIGIVELGGGYRTADLDSYFSSLGVGSPKVVAVGVDQSGNQPTGDPNGPDGEVTLDIEIAGALAPAATIAVYFTTNSDAGFIDAVSQAVHDRTNQPSVISISWGAPESMWTAQSMKALNDVLQSAAAIGVTVCAASGDSGSSDGVGDGRDHVDFPASSPYVLACGGTSLQGSGRTVAHEVVWNDGSNGGATGGGVSGAFPVPAWQEGLSTSAAQGGQRALTGRGVPDVAGDASPLTGYDVIVDGNNTVIGGTSAVAPLWAALIARINGAKGAPVGFVNPKLYKASACNDITQGNNGSYAATTGWDACTGLGSPDGVKVAAAL

SEQ ID NO: 107 - protein, 512 aa, synthetic construct; identical to SEQ ID NO: 84
  REGION 1..512: note = Synthetic

SEQ ID NO: 108 - protein, 531 aa, synthetic construct; identical to SEQ ID NO: 85
  REGION 1..531: note = Synthetic

SEQ ID NO: 109 - protein, 567 aa, synthetic construct; identical to SEQ ID NO: 86
  REGION 1..567: note = Synthetic

SEQ ID NO: 110 - protein, 543 aa, synthetic construct; identical to SEQ ID NO: 87
  REGION 1..543: note = Synthetic

SEQ ID NO: 111 - protein, 378 aa, synthetic construct
  REGION 1..378: note = Synthetic
MHSYLKQQSHMQSYLEQENHMRSYLEMRKKPYFDDLANIRPGGLTPAQVCQAYQFAKVQPVRPVKLGIVSLAGQYLSSDMSKAFTGYGLPTPVVSTAGSQVLGDLWSNVENMMDIEIAGAAWAYATGTAATLLMQFEPNNETGIPNAINALVAAGCEVISISWGAPANLQTMEAITARKEACKQAAVQNVHVFAASGDESLNDGTNSRTPDDPCCDPNVWGVGGTRLVLQADGSIAQESAWGDGNAADKGGGGGFDSREPLPDYQVGVVHSEHRGSPDSSANADPGTGYAIVANGQWLIGGGTSASAPLTAGYVAAILSTLPGPISQSVLQRKLYTAHKTAFRDILLGSNGAPARPGWEEATGLGSINGPGLAAALQS

SEQ ID NO: 112 - protein, 523 aa, synthetic construct; identical to SEQ ID NO: 89
  REGION 1..523: note = Synthetic